(19) United States
(12) Patent Application Publication
(10) Pub. No.: US 2012/0064008 A1
(43) Pub. Date: Mar. 15, 2012

(54) COMPOSITIONS FOR THE TREATMENT OF METASTATIC CANCER AND METHODS OF USE THEREOF

(76) Inventors: Bruce Zetter, Wayland, MA (US); Ivy Chung, Boston, MA (US); Courtney Barrows, Danvers, MA (US)

(21) Appl. No.: 13/320,635

(22) PCT Filed: May 20, 2010
(86) PCT No.: PCT/US10/35578

§ 371 Date: Nov. 15, 2011

Related U.S. Application Data
(60) Provisional application No. 61/180,029, filed on May 20, 2009.

(51) Int. Cl. A61K 49/00; A61P 35/04; A01K 67/00; A61K 31/4184

(52) U.S. Cl. 424/9.2; 514/394; 514/395; 800/10

(57) ABSTRACT

An in vivo assay for assessing the metastatic potential of cancer cells has been developed. This is a functional assay that also allows for screening of compounds that are selective for metastatic cancer. Metastatic cancer is established in animals by intravenous injection of metastatic cancer cells before treatments are tested. This assay has been used to identify benzimidazoles for the treatment of metastatic prostate cancers. The benzimidazole(s) can be formulated for enteral or parenteral administration. In one embodiment, the compositions are formulated for parenteral administration. Compositions containing a benzimidazole exhibit greater cytotoxicity against cell lines prone to metastasis than against cells lines that are less prone to metastasis in vitro. The benzimidazoles described herein were effective at treating metastatic prostate cancer in bone. Administration of benzimidazoles in metastatic prostate cancer mouse models showed a significant increase in the survival times of the animals, even against paclitaxel resistant cancer cell lines.

### FIELD OF THE INVENTION

The invention is in the field of pharmaceutical compositions for the treatment of cancer and methods of using thereof, specifically compositions for the treatment of metastatic cancers and methods of use thereof.

### BACKGROUND OF THE INVENTION

Metastatic cancer is cancer that has spread from its primary site (the part of the body in which it developed) to other parts of the body. If cells break away from a cancerous tumor, they can travel to other areas of the body. The spread of a tumor to a new part of the body is called metastasis.

It is important to understand the difference between metastasis and local spread, because they affect a patient's prognosis and treatment options. Local spread means that a growing cancer extends beyond the organ in which it developed, into nearby organs and tissues. For example, the cervix (lower part of the uterus or womb) is located in front of the rectum and behind the bladder. Very large cancers of the cervix may extend into the rectum or bladder.

Metastasis involves spread of cancer cells through the bloodstream, or the lymph system. Cancer cells that break off from tumors and enter the lymph vessels may be carried to lymph nodes where they may continue to grow and form metastases. Metastasis to lymph nodes near the place a cancer developed is sometimes referred to as regional spread. This is to distinguish it from distant spread or distant metastasis. Distant spread generally occurs when cancer cells break off from tumors and enter the bloodstream, travel to other organs, and continue to grow into new tumors.

Most people who die of cancer have metastases at the time of their death. These metastases are directly responsible for the majority of cancer deaths. Most common cancers (prostate, breast, colon, lung, for example) develop in organs that can be completely or partially removed by surgery. Surgery could cure patients if metastasis did not occur. Most of the serious consequences of these cancers occur because of spread to other parts of the body. In some cases, the most serious effect of cancer is its spread to a particularly essential part of the body, such as areas of the brain, lungs and lymph nodes. In other cases, spread to and growth in many organs creates so many cancer cells that the body's normal metabolism is disrupted.

Metastasis can be affected by different variables. For example, cells can be released into the bloodstream during a surgical procedure, resulting in metastasis to distant sites. Metastasis can also result from the cancer being of a more aggressive type than other types of the same kind of cancer. Other factors have been proposed, but are not established. Cancers that are likely to be metastatic may be indicated by the presence of specific markers or the “grade” given to the tumor upon histology. For example, in prostate cancer a Gleason score of 6 or higher is indicative of metastatic cancer.

Prostate cancer is a disease in which cancer develops in the prostate, a gland in the male reproductive system. Rates of prostate cancer vary widely across the world. Although the rates vary widely between countries, it is least common in South and East Asia, more common in Europe, and most common in the United States. Prostate cancer develops most frequently in men over fifty. It is the most common type of cancer in men in the United States, where it is responsible for more male deaths than any other cancer, except lung cancer. In the United Kingdom it is also the second most common cause of cancer death after lung cancer, where around 35,000 cases are diagnosed every year, 10,000 of which are fatal.

Prostate cancer is characterized by its stage of development using the four-stage tumor/nodes/metastases (TNM) system. Its criteria include the size of the tumor, the number of involved lymph nodes, and the presence of any metastases. Prostate cancers defined as T3 or T4 indicate the cancer has metastasized and spread to other organs/tissues. The most common metastases are to the lung; bone, particularly the pelvis, spine, and ribs; the liver, and/or the lymph nodes.

The treatment for metastatic prostate cancer depends on a variety of factors including the size of the tumor(s), the extent to which the cancer has spread to other part of the body, the treatments already performed, and the patient's overall physical condition. Some of the treatment options, which may be used alone or in combination, include orchidectomy (removal of one or both testicles to reduce the amount of testosterone that is produced); treatment with hormones or hormone antagonists; chemotherapy; and radiotherapy. However, these treatments have varying rates of success and often result in adverse side effects which significantly reduce the quality of life for the patient.

Therefore, it is an object of the invention to provide compositions for the treatment of metastatic cancer, particularly metastatic prostate cancer, and methods of screening for and using thereof.

It is another object of the present invention to provide an assay for determining the likelihood of a cancer becoming metastatic.

It is still another object of the present invention to provide formulations for treating metastatic cancers, especially prostate cancer.

### SUMMARY OF THE INVENTION

An in vivo assay for assessing the metastatic potential of cancer cells has been developed. This is a functional assay that also allows for screening of compounds that are selective for metastatic cancer. Metastatic cancer is established in animals by intravenous injection of metastatic cancer cells. The cancer cells are allowed to become established before treatments are tested.

This assay has been used to reveal the utility of benzimidazoles for the treatment of metastatic prostate cancers. In one embodiment, the prostate cancer is disseminated, hormone-refractory prostate cancer. Compositions for systemic administration containing one or more benzimidazoles, such as the antihelmintic benzimidazoles, have been developed. These benzimidazoles include fenbendazole, albendazole, flubendazole, oxbendazole, mebendazole, enbendazole, albendazole sulfone, rycobendazole, thiabendazole, oxfendazole, flubendazole carbendazim, benomyl, thiabendazole, thiosphanate, derivatives and analogs thereof, prodrugs thereof, and combinations thereof. The compositions can further contain one or more additional active agents to enhance the efficacy of the compositions, such as paclitaxel or another taxane. In one embodiment, the purified enantiomer of the compound is used. In another embodiment, a mixture of enantiomers is used. The compositions can be formulated for controlled release, such as delayed release, sustained release, pulsatile release, or combinations thereof.

The compositions can be formulated for enteral or parenteral administration. In one embodiment, the compositions are formulated for parenteral administration. In a preferred embodiment, the benzimidazole is dissolved in an organic solvent and surfactant carrier. These compounds are extremely hydrophobic and insoluble, unless modified or formulated to increase solubility. Formulations suitable for other hydrophobic insoluble drugs such as taxol can be utilized. For example, in one embodiment, the benzimidazole is formulated in a mixture of dimethyl sulfoxide (DMSO), N-methyl-2-pyrrolidone (NMP), Tween®-80, and Cremophor EL in a ratio of 1:3:2:2 (the system as a whole is referred to as “DNTC”).

In the embodiment where the composition is formulated for parenteral administration, controlled release formulations can be prepared by incorporating the benzimidazole into nanoparticles, microparticles, or combinations thereof, wherein the particles are formed by one or more materials that control release of the drug such as natural and/or synthetic polymers, waxes, and fats. The particles can also be coated with one or more controlled release coatings. The compositions can be formulated for immediate release, delayed release, extended release, pulsatile release, and combinations thereof.

Compositions containing a benzimidazole exhibit greater cytotoxicity against cell lines prone to metastasis than against cells lines that are less prone to metastasis in vitro. For example, fenbendazole solubilized in DNTC showed increased cytotoxicity against prostate cancer cells compared to those solubilized in DMSO, based on ED 50 (defined as the dose that kills 50% of the cell population) when tested in two human prostate cancer cell lines known to metastasize, PC-3M and the more aggressive PC-3MLN4. Bioavailability studies indicate a 10-fold increase in benzimidazole metabolites in DNTC formulations compared to DMSO.

The benzimidazoles described herein were effective at treating metastatic prostate cancer in bone. In a model of experimental bone metastasis, animals treated with benzimidazole showed reduced tumor growth compared to those treated with vehicle. Vehicle-treated mice had extensive osteolysis due to the osteoclastic bone resorption activity of PC-3MLN4 cells. In contrast, bone integrity was maintained in benzimidazole-treated mice.

Administration of benzimidazoles in metastatic prostate cancer mouse models showed a significant increase in the survival times of the animals, even against paclitaxel resistant cancer cell lines. This is significant since most men with metastatic prostate lesions who fail hormone deprivation therapy and under go paclitaxel-based chemotherapy have a mean survival rate of two months.

### BRIEF DESCRIPTION OF THE DRAWINGS

FIGS. 1A-1C are graphs comparing the cytotoxicity of benzimidazoles (percent control) dissolved in dimethylsulfoxide (DMSO, 12.5% by weight of the carrier), N-methyl-2-pyrrolidone (NMP, 37.5% by weight of the carrier), TWEEN-80 (25% by weight of the carrier), and Cremophor® EL (25% by weight of the carrier), referred to as the combination carrier or DNTC, and DMSO alone against PC3M and PC3MLN 4 cell lines. FIG. 1A compares the cytotoxicity of fenbendazole (μM) dissolved in the combination carrier (open diamonds/solid line) and DMSO (open diamonds/dotted line) against PC3M cells and fenbendazole (μM) dissolved in the combination carrier (solid circle/solid line) and DMSO (solid circle/dotted line) against PC3MLN4 cells. FIG. 1B compares the cytotoxicity of albendazole (μM) dissolved in the combination carrier (open diamonds/solid line) and DMSO (open diamonds/dotted line) against PC3M cells and albendazole (μM) dissolved in the combination carrier (solid circle/solid line) and DMSO (solid circle/dotted line) against PC3MLN4 cells. FIG. 1C shows the cytotoxicity of the combination carrier (μM) against PC3M cells (open diamonds/solid line) and PC3MLN4 cells (solid circles/solid line).

FIGS. 2A-2D are graphs comparing the cytotoxicity (relative fluorescence) by fraction compared to control of various benzimidazoles (μM) against PC3M cells and PC3MLN4 cells. FIG. 2A is fenbendazole; FIG. 2B is albendazole; FIG. 2C is carbendazim; and FIG. 2D is benomyl.

FIGS. 3A-3H are graphs showing the effect of various benzimidazoles on apoptosis in PC3M (FIGS. 3A-3D) and PC3MLN4 (FIGS. 3E-3H) cell lines. FIGS. 3A and 3E are the combination carrier; FIGS. 3B and 3F are 1 μM fenbendazole; FIGS. 3C and 3G are 1 μM albendazole; and FIGS. 3D and 3H are 1 μM mebendazole.

FIG. 4A is a graph showing percent apoptosis in PC-3M and PC-3 mLN4 cells as a function of benzimidazole. Percent apoptosis was determined from flow cytometry analysis of annexin V-stained cells, 72 hours after treatment, with drug or with vehicle control *, P<0.001, compared to PC-3M. FIG. 4B is a graph showing percent apoptosis in PC-3M and PC-3 mLN4 cells as a function of benzimidazole after treatment of the cells with caspase-3 inhibitor (Z-VAD-FMK). Percent apoptosis was determined from flow cytometry analysis of annexin V-stained cells, 72 hours after treatment, with drug or with vehicle control *, P<0.001, relative to cells treated with vehicle only.

FIG. 5 is a graph comparing the survival time (days) of prostate cancer metastasis-bearing animals treated with combination carrier+saline, 50 or 100 mg/kg albendazole, 50 or 100 mg/kg mebendazole, and 50 mg/kg oxibendazole.

FIG. 6 is a graph comparing the survival time (days) of prostate cancer metastasis-bearing animals treated with combination carrier+saline, 100 mg/kg albendazole, 100 mg/kg mebendazole, and 100 mg/kg fenbendazole.

FIG. 7 is a graph comparing the survival time (days) of prostate cancer metastasis-bearing mice treated with vehicle (control), 10 mg/kg paclitaxel, 100 mg/kg albendazole, 250 mg/kg albendazole, and 100 mg/kg benomyl.

FIG. 8 is a graph comparing tumor burden in animals treated with combination carrier+saline, 50 or 100 mg/kg albendazole, 50 or 100 mg/kg mebendazole, and 50 mg/kg oxibendazole.

FIG. 9 is a graph comparing tumor burden in animals treated with combination carrier+saline, 100 mg/kg albendazole, 100 mg/kg mebendazole, and 100 mg/kg fenbendazole.

FIG. 10 is a graph comparing tumor cell proliferation (Ki67 labeling index) in animals treated with combination carrier plus saline and 50 g/kg fenbendazole.

FIGS. 11A-11C are graphs showing the cytotoxic effect of a combination of benzimidazole and paclitaxel. FIG. 11A is fenbendazole plus paclitaxel; FIG. 11B is albendazole+paclitaxel; and FIG. 11C is mebendazole plus paclitaxel.

FIGS. 12A-12D are graphs comparing the cytotoxic effect (percent control) of paclitaxel (♦), fenbendazole (□), and albendazole (∘) against paclitaxel resistant PC3-TR (FIG. 12B) and DU145-TR (FIG. 12D) cell lines and their paclitaxel-sensitive counterparts PC3 (FIG. 12A) and DU 145 (FIG. 12C) cell lines. FIG. 12E is a graph showing the relative tumor growth of PC-3TxR cells injected subcutaneously in mice after treatment with DNTC vehicle, paclitaxel, FBZ or MBZ. Treatment was given three times per week for three weeks. FIG. 12F is a graph showing the average tumor size from each treatment group on the day of sacrifice. N=10 per group; P<0.01.

FIG. 13 is a graph showing the mean luciferase signals (p/s/cm²/sr) detected in mice during the course of treatment as a measurement of tumor burden.

FIGS. 14A-J are graphs showing the cytotoxicity of fenbendazole, albendazole or mebendazole against various other human cancer types including ACHN human renal cell carcinoma cells (FIG. 14A), U2OS human osteosarcoma cells (FIG. 14B), AsPC-1, BxPC-3 and Capan-2 human pancreatic adenocarcinoma cells (FIGS. 14C-E), HT1080 human fibrosarcoma cells (FIG. 14F), MESSA human uterine sarcoma (FIG. 14G), MCF-7 human breast cancer cells (FIG. 14H), A549 human lung adenocarcinoma cells (FIG. 14I) and H460 human non-small cell lung cancer cells (FIG. 13J). The active agents were formulated in the combination carrier. The cells were treated for 72 hours. Percent control was calculated by dividing the fluorescent reading (Cyquant assay) from drug-treated cells with those from vehicle-treated cells, indicating percentage of cell survival

### DETAILED DESCRIPTION OF THE INVENTION

### I. Definitions

“Tumor grade” is a system used to classify tumors in terms of how abnormal they look under a microscope and how quickly the tumor is likely to grow and spread. Many factors are considered when determining tumor grade, including the structure and growth pattern of the cells. The specific factors used to determine tumor grade vary with each type of cancer.

“Histologic grade”, also called differentiation, refers to how much the tumor cells resemble normal cells of the same tissue type. Nuclear grade refers to the size and shape of the nucleus in tumor cells and the percentage of tumor cells that are dividing. Based on the microscopic appearance of cancer cells, pathologists commonly describe tumor grade by four degrees of severity: Grades 1, 2, 3, and 4. The cells of Grade 1 tumors resemble normal cells and tend to grow and multiply slowly. Grade 1 tumors are generally considered the least aggressive in behavior.

Conversely, the cells of Grade 3 or Grade 4 tumors do not look like normal cells of the same type. Grade 3 and 4 tumors tend to grow rapidly and spread faster than tumors with a lower grade.
The American Joint Commission on Cancer recommends the following guidelines for grading tumors (1):

| Grade | |
|---|---|
| GX | Grade cannot be assessed (Undetermined grade) |
| G1 | Well-differentiated (Low grade) |
| G2 | Moderately differentiated (Intermediate grade) |
| G3 | Poorly differentiated (High grade) |
| G4 | Undifferentiated (High grade) |

Grading systems are different for each type of cancer. For example, pathologists use the Gleason system to describe the degree of differentiation of prostate cancer cells. The Gleason system uses scores ranging from Grade 2 to Grade 10. Lower Gleason scores describe well-differentiated, less aggressive tumors. Higher scores describe poorly differentiated, more aggressive tumors. Other grading systems include the Bloom-Richardson system for breast cancer and the Fuhrman system for kidney cancer.

“Cancer stage” refers to the extent or severity of the cancer, based on factors such as the location of the primary tumor, tumor size, number of tumors, and lymph node involvement (spread of cancer into lymph nodes).

“Metastatic cancer”, as used herein, refers to a primary cancer capable of metastasis or secondary cancer or secondary cancers which have metastasized from a primary cancer. Metastatic cancer also refers to tumors defined as being high grade and/or high stage, for example tumors with a Gleason score of 6 or higher in prostate cancer are more likely to metastasize. Metastatic cancer also refers to tumors defined by one or more molecular markers that correlate with the production of metastasis.

“Highly Metastatic cancer cells” are those cells that form significantly more, for example at least twice as many, visible (greater than 1 mm in diameter) metastasis on the surface of an organ compared to cells with low metastatic potential, wherein significance is revealed by a P value less than 0.05 by a statistical test, such as the student's T-test.

“Metastatic tumors”, as used herein, refers to secondary tumors, secondary cancerous tissue, and/or secondary cancerous cells which metastasized from cancerous tissues and/or cells in primary or secondary tumors that are able to metastasize.

“Antihelmintic benzimidazole”, as used herein, refers to benzimidazole compounds which exhibit antihelmintic activity.

“Benzimidazole”, as used herein, refers to compounds containing a benzimidazole core. The compounds may or may not have antihelmintic activity.

“Derivative” and “analog” are used interchangeably, and refer to a compound or compounds derived from another compound. For example, “derivative(s) of albendazole” refers to a compound or compounds derived from albendazole. Derivations include, but are not limited to, replacement of one or more functional groups with another functional group; deletion of a functional group, addition of a functional group, and combinations thereof. In a preferred embodiment, the derivative or analog retains the core of the compound from which it is derived. For example, preferred “derivatives or analogs of albendazole” retain the benzimidazole core and differ in the number and/or identity of the substituents on the benzimidazole core.

“Alkyl”, as used herein, refers to the radical of saturated or unsaturated aliphatic groups, including straight-chain alkyl, alkenyl, or alkynyl groups, branched-chain alkyl, alkenyl, or alkynyl groups, cycloalkyl, cycloalkenyl, or cycloalkynyl(alicyclic) groups, alkyl substituted cycloalkyl, cycloalkenyl, or cycloalkynyl groups, and cycloalkyl substituted alkyl, alkenyl, or alkynyl groups. Unless otherwise indicated, a straight chain or branched chain alkyl has 30 or fewer carbon atoms in its backbone (e.g., C₁-C₃₀ for straight chain, C₃-C₃₀ for branched chain), preferably 20 or fewer, preferably 10 or fewer, more preferably 6 or fewer, most preferably 5 or fewer. If the alkyl is unsaturated, the alkyl chain generally has from 2-30 carbons in the chain, preferably from 2-20 carbons in the chain, preferably from 2-10 carbons in the chain, more preferably from 2-6 carbons, most preferably from 2-5 carbons. Likewise, preferred cycloalkyls have from 3-20 carbon atoms in their ring structure, preferably from 3-10 carbons atoms in their ring structure, most preferably 5, 6 or 7 carbons in the ring structure. Examples of saturated hydrocarbon radicals include, but are not limited to, methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl. Examples of unsaturated alkyl groups include, but are not limited to, vinyl, 2-propenyl, crotyl, 2-isopentenyl, 2-(butadienyl), 2,4-pentadien yl, 3-(1,4-pentadienyl), ethynyl, 1- and 3-propynyl, and 3-butynyl.

The term “alkyl” includes one or more substitutions at one or more carbon atoms of the hydrocarbon radical as well as heteroalkyls. Suitable substituents include, but are not limited to, halogens, such as fluorine, chlorine, bromine, or iodine; hydroxyl; —NR₁R₂, wherein R₁ and R₂ are independently hydrogen, alkyl, or aryl, and wherein the nitrogen atom is optionally quaternized; —SR, wherein R is hydrogen, alkyl, or aryl; —CN; —NO₂; —COON; carboxylate; —COR, —COOR, or —CONR₂, wherein R is hydrogen, alkyl, or aryl; azide, aralkyl, alkoxyl, imino, phosphonate, phosphinate, silyl, ether, sulfonyl, sulfonamido, heterocyclyl, aromatic or heteroaromatic moieties, —CF₃; —CN; —NCOCOCH₂CH₂; —NCOCOCHCH; —NCS; and combinations thereof.

“Aryl”, as used herein, refers to C₅-C₁₀-membered aromatic, heterocyclic, fused aromatic, fused heterocyclic, biaromatic, or bihetereocyclic ring systems. Broadly defined, “aryl”, as used herein, includes 5-, 6-, 7-, 8-, 9-, and 10-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amino, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN; and combinations thereof.

The term “aryl” also includes polycyclic ring systems having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxa zolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromenyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined above for “aryl”.

The terms “alkenyl” and “alkynyl” refer to unsaturated aliphatic groups analogous in length and possible substitution to the alkyls described above, but that contain at least one double or triple bond respectively.

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

“Alkoxy”, “alkylamino”, and “alkylthio” are used to refer to those alkyl groups attached to the remainder of the molecule via an oxygen atom, an amino group, or a sulfur atom, respectively.

“Alkylaryl”, as used herein, refers to an alkyl group substituted with an aryl group (e.g., an aromatic or hetero aromatic group).

“Heterocycle” or “heterocyclic”, as used herein, refers to a cyclic radical attached via a ring carbon or nitrogen of a monocyclic or bicyclic ring containing 3-10 ring atoms, and preferably from 5-6 ring atoms, consisting of carbon and one to four heteroatoms each selected from the group consisting of non-peroxide oxygen, sulfur, and N(Y) wherein Y is absent or is H, O, (C1-10)alkyl, phenyl or benzyl, and optionally containing 1-3 double bonds and optionally substituted with one or more substituents. Examples of heterocyclic ring include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Heterocyclic groups can optionally be substituted with one or more substituents as defined above for alkyl and aryl.

“Halogen”, as used herein, refers to fluorine, chlorine, bromine, or iodine.

“Stereoisomer”, as used herein, refers to isomeric molecules that have the same molecular formula and sequence of bonded atoms (constitution), but which differ in the three dimensional orientations of their atoms in space. Examples of stereoisomers include enantiomers and diastereomers. As used herein, an enantiomer refers to one of the two mirror-image forms of an optically active or chiral molecule. Diastereomers (or diastereoisomers) are stereoisomers that are not enantiomers (non-superimposable mirror images of each other). Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2n, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Enantiomer can be resolved or separated using techniques known in the art.

“Prochiral”, as used herein, generally refers to molecules which can be converted from achiral (not chiral) to chiral in one or more steps. For examples, prochiral molecules can be converted to chiral molecules through metabolism in organisms, such as plants, animals, bacteria, etc.

“Prodrug”, as used herein, refers to an active drug chemically transformed into a per se inactive derivative which, by virtue of chemical or enzymatic attack, is converted to the parent drug within the body before or after reaching the site of action. Prodrugs are frequently (though not necessarily) pharmacologically inactive until converted to the parent drug. Exemplary prodrugs include, but are not limited to, esters, amides, polyethylene glycol prodrugs, N-acyl amines, dihydropyridine prodrugs, polypeptide prodrugs; 2-hydroxybenzamides, carbamates, carbamates with simple built-in hydrolysis features, N-oxides that biologically reduced to the parent amine, N-mannich base prodrugs, and prodrugs containing Schiff bases.

“Parenteral administration”, as used herein, means administration by any method other than through the digestive tract or non-invasive topical or regional routes. For example, parenteral administration may include administration to a patient intravenously, intradermally, intraarterially, intraperitoneally, intralesionally, intracranially, intraarticularly, intraprostatically, intrapleurally, intratracheally, intravitreally, intratumorally, intramuscularly, subcutaneously, subconjunctivally, intravesicularly, intrapericardially, intraumbilically, by injection, and by infusion.

### I. Assay for Identification of Drugs Preferentially Killing Metastatic Tumors or Cancer Cells

The animal model described in the examples is useful for screening for compounds that are more effective against metastatic cancers. Many cancer drugs are ineffective when used to treat patients with metastatic disease, implying that metastatic tumor cells may differ from non-metastatic or poorly metastatic cells in their response to particular drugs. The assay described herein is designed to detect drugs or treatments that are preferentially effective against metastatic tumor cells. A “hit” exhibits less than 50% of the LD50 when tested on nBE cells relative to the highly metastatic tumor cells. The assay relies on sets of highly related cell lines that differ in their relative ability to form metastases in experimental animals. A “hit” was denoted as a drug or agent that demonstrated significantly greater cytotoxicity or inhibition of cell proliferation in cell culture when applied to highly metastatic tumor cells relative to their less metastatic counterparts. The animal model and screen described herein can be modified to screen for compounds effective at treating a variety of metastatic cancers.

In one embodiment, the metastatic cancer is metastatic prostate cancer. The design of the in vitro screening allows for a systematic screening of candidate drugs using both human and animal metastatic prostate cancer models that encompass various genetic backgrounds but share similar phenotypic characteristics with hormone-refractory, metastatic prostate cancer disease in humans.

The cytotoxic effects of 1120 compounds from the Prestwick chemical drug library were evaluated in several phases of in vitro screening. In the first phase, drugs were selected based on preferential toxicity against highly metastatic human prostate cancer cells PC-3MLN4 relative to the less aggressive counterpart, PC-3M (9). In contrast to PC-3M, PC-3MLN4 cells are highly metastatic to lymph nodes when implanted in the mouse prostate. Cells were treated with a fixed concentration (10 μM) for 48 hours and analyzed for remaining viable cells. A drug was considered a “hit” if it resulted in a) a significantly greater growth inhibition in PC-3MLN4 than in PC-3M cells; or b) at least 80% growth inhibition in both of the cell lines. 44 of the drugs were identified as “hits” and were taken into the second line of screening, in which these drugs were tested in a range of concentration for 48 hours in PC-3M/PC-3MLN4 (Screen 1), as well as in a second pair, DU-145/DU-145LN4 (Screen II). Similar to PC-3MLN4 cells, DU-145LN4 is a metastatic variant established from lymph node metastases after injection of DU-145 cells into the mouse prostate. These prostate cancer cells are androgen receptor negative as is frequently the case in hormone refractory prostate cancer disease. “Hits” from this screen were defined when a drug showed preferential cytotoxicity in both of the metastatic variants with at least 80% growth inhibition and a significant difference (p<0.05) when compared to the parental counterpart, for at least two of the concentrations tested. 23 drug candidates (2% of the total library) were considered a “hit” in this screen.

As described in the example, potential drug candidates were evaluated in a highly aggressive Dunning rat prostatic adenocarcinoma AT6.1 model, which is also androgen-unresponsive and develops lymph node and lung metastases when the rats are inoculated subcutaneously in rats (Screen III). In this screen, cells were treated with drugs over a range of concentration for 48 hours, and “hits” were identified when there is less than 40% growth inhibition in at least two concentrations tested.

To further increase the stringency of the screening process, the drug candidates were then tested for minimal cytotoxic effects to normal host cells (e.g., more than 90% survival) using a non-tumorigenic rat epithelial nBE cells (Screen IV).

From this library, candidate drugs were identified that selectively exert cytotoxic effects on all metastatic prostate cancer cell lines tested but have reduced cytotoxic effects on normal rat epithelial cells. This assay can be similarly used to identify anti-metastatic agents for other types of tumors.

### II. Compounds

Screens, including an animal model, for identifying compounds effective at treating metastatic cancers are described herein. In one embodiment, an in vitro screen of metastatic prostate cancer identified a class of compounds known as benzimidazoles which are preferentially cytotoxic to metastatic cancer cells. This activity was confirmed using an animal model of metastatic prostate cancel as described in the Examples.

A. Benzimidazoles

In one embodiment, the compositions described herein contain one or more benzimidazoles in an amount effective to reduce or prevent the growth or proliferation of metastatic cancer cells or tumors derived therefrom. These compounds have been shown to preferentially kill human metastatic tumor cells and extend the lives of animals with metastatic cancers as illustrated in the examples.

In one embodiment, the benzimidazole has the following chemical formula:

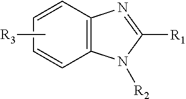

wherein

R₁ is selected from H; alkyl; alkenyl; alkynyl; carboxyl (—CO₂H); hydroxyl; alkoxy, amino; alkyl amino, dialkylamino; halogen, such as chloro; haloalkyl, such a mono, di, or trihaloalkyl; haloalkoxy, such as mono, di, or trihaloalkoxy (e.g., difluormethoxy); aryl, such as phenyl; benzoyl; aryl-thio, such as phenyl-thio; heteroaryl, such as pyridinyl; alkyl-thio, such as propyl-thio; diaryl, such as diphenyl; SH; carbamate (e.g., —NHCOOR₅, wherein R₅ is substituted or unsubstituted alkyl, alkenyl, alkynyl, aryl, or heteroaryl); piperidin-4-yl; 3-thiophenyl; and esters (—CO₂R₄) wherein R₄ is selected from the group that includes alkoxy, haloalkyl, alkenyl, and cycloalkyl; and

R₂ is hydrogen, —CO₂(alkyl), —CO₂(cholester-3-yl), CO₂(CH₂)ₘCOOH, —CO₂(CH₂)ₘCO₂(alkyl), α-methylvinyl, 3-chloropropyl or piperidin-4-yl, wherein in is 1 to 10, and alkyl is preferably C₁-C₆ alkyl;

R₃ is selected from H; alkyl; alkenyl; alkynyl; carboxyl (—CO₂H); hydroxyl; alkoxy, amino; alkyl amino, dialkylamino; halogen, such as chloro; haloalkyl, such a mono, di, or trihaloalkyl; haloalkoxy, such as mono, di, or trihaloalkoxy (e.g., difluormethoxy); aryl, such as phenyl; benzoyl; aryl-thio, such as phenyl-thio; heteroaryl, such as pyridinyl; alkyl-thio, such as propyl-thio; diaryl, such as diphenyl; methoxy(methoxy-dimethyl, pyridinyl)methyl-(sulfonyl); fluorophenylmethyl-2-chloro; propenyl; chloropropyl; or esters (—CO₂R₄) wherein R₄ is selected from the group that includes alkoxy, haloalkyl, alkenyl, and cycloalkyl, wherein the alkyl groups have from 1-8 carbons, or CH₃CH₂(OCH₂CH₂)ₙ—, or CH₃CH₂CH₂(OCH₂CH₂CH₂)ₙ, or (CH₃)₂CH(OCH(CH₃)CH₂)ₙ—, wherein n is from 1-3;

or the pharmaceutically effective organic or inorganic salts thereof, or mixtures thereof.

In a preferred embodiment, R₂ is hydrogen and R₁ and R₃ are as defined above.

In another embodiment, the benzimidazole has the formula:

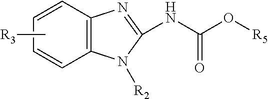

wherein R₂ and R₃ are as defined above and R₅ is substituted or unsubstituted alkyl, alkenyl, or alkynyl. In one embodiment, R₅ is substituted or unsubstituted C₁-C₄ alkyl.

Suitable benzimidazoles include, but are not limited to, fenbendazole, albendazole, flubendazole, oxibendazole, mebendazole, enbendazole, albendazole sulfone, rycobendazole (ricobendazole), thiabendazole, oxfendazole, flubendazole carbendazim, benomyl, thiabendazole, thiosphanate, analogs and derivatives thereof, prodrugs thereof, and combinations thereof. Although these may be referred to herein as antihelmintic benzimidazoles, it will be understood that not all of the drugs will be approved for, or effective clinically in, treating of helmintic infections.

In one embodiment, the benzimidazole is fenbendazole or a derivative or prodrug thereof. Fenbendazole is a broad spectrum benzimidazole anthelmintic used against gastrointestinal parasites including roundworms, hookworms, whipworms, the taenia species of tapeworms, pinworms, aelurostrongylus, paragonimiasis, strongyles and strongyloides. Exemplary derivatives of fenbendazole include, but are not limited to, oxfendazole, fenbendazole sulfone, and combinations thereof. In another embodiment, the benzimidazole is albendazole or a derivative or prodrug thereof. In still another embodiment, the benzimidazole is mebendazole or a derivative or prodrug thereof. Structures of representative benzimidazoles are shown below:

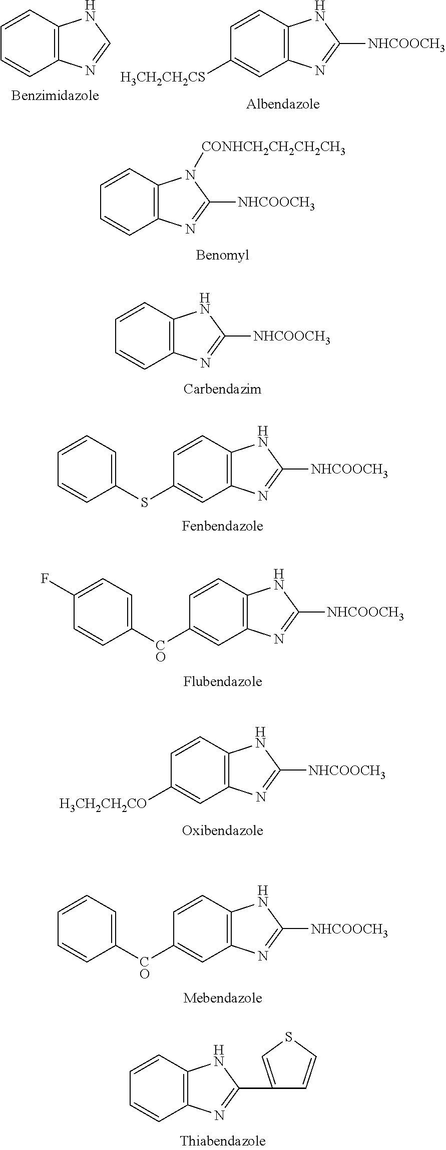

Additional benzimidazoles are disclosed in U.S. Pat. Nos. 5,538,990; 5,475,005; 5,468,765; 5,459,155; and 4,436,737, which are incorporated herein by reference where appropriate.

The benzimidazole may be chiral and may be formulated as a single stereoisomer or a mixture of stereoisomers, for example, as a single enantiomer or a mixture of enantiomers. If the benzimidazole is formulated as a mixture of enantiomers, the mixture may be a racemic mixture (i.e., a 50/50 mixture by weight of enantiomers) or an enantiomerically enriched mixture, wherein one enantiomer is present in an amount greater than 50% by weight. The dose of the benzimidazole administered can be varied based on whether a single enantiomer is administered or a mixture of enantiomers is administered.

Some benzimidazoles are prochiral. For example, albendazole and fenbendazole contain sulfur atoms which are achiral centers. However, albendazole and fenbendazole can undergo enantioselective sulfoxidation in vivo to form chiral sulfoxides. Virkel et al., Drug. Metab. Dispos., 32(5), 536-544 (2004) describes the enantioselective sulfoxidation of albendazole and fenbendazole, to albendazole sulfoxide and oxfendazole, respectively, in liver, lung, and small intestine microsomes obtained from healthy sheep and cattle as well as by liver microsomal mixed function oxidases in rats. Albendazole sulfoxide and oxfendazole are the main anthelmintically active metabolic products found systemically after albendazole and fenbendazole administration to sheep and cattle. Albendazole and fenbendazole are converted to their (+) and (−) sulfoxide enantiomers in vivo; however, the (+) enantiomer of the sulfoxide enantiomers appears to be the predominant enantiomer in plasma, as well as in parasitic locations such as the lung and GI mucosa, of sheep and cattle. In view of the prochirality of some benzimidazoles, formulations containing a single stereoisomer or a mixture of stereoisomers of the chiral metabolite or metabolites can also be used.

Other antihelmintic but non-benzimidazole drugs that may be useful include niclosamide, pyrvinium pamoate, and quinacrine dihydrochloride dihydrate.

Drugs that are useful in preferential killing of metastatic cancer cells can be selected using the in vivo assay described in the examples. In this assay, unlike most animal models of cancer therapy, the cancer cells are injected intravenously and allowed to spread and form tumors throughout the animal, prior to administration of the drug. The cancer cells are preferably a cell line that is known to be highly metastatic. The preferred animal is a rat or mouse, and the cells are allowed to engraft for about five to eight days before treatment is initiated to allow for metastatic colonization. Tumor cells may also be injected into the skin, prostate, or other internal organs and allowed to metastasize before drug treatment begins. Genetically-altered animals that develop spontaneous metastatic prostate tumors may also be utilized. Drug is usually administered by injection, intravenously or intraperitoneally, but may be administered orally or by depo.

Potential drug candidates exhibiting selective anti-metastatic tumor activity were identified by screening the 1120 compounds of the Prestwick Chemical Drug Library using the in vitro assays described in the examples. In the first and second phase of screening, drugs were selected based on a preferential toxicity in highly metastatic human prostate cancer cells PC-3MLN4, DU145LN4, and DU-145LU4 compared to their less aggressive counterparts, PC-3M and DU-145.

In one embodiment, the benzimidazole is fenbendazole, albendazole and/or mebendazole, derivatives or analogues thereof, prodrugs thereof, or combinations thereof. The compound may be formulated as a pharmaceutically acceptable salt.

Fenbendazole, albendazole and mebendazole are all benzimidazole methylcarbamates, one of the four types of benzimidazoles utilized as anti-parasitic and anti-fungal agents. These drugs are generally well tolerated and lack broad systemic toxicity, a preferred drug property for cancer patients. Although not widely recognized as anti-cancer drugs, they have been employed sporadically as anti-tumor agents in patients with hepatocellular carcinoma, peritoneal carcinomatosis, and ovarian cancer. Most of these studies used these drugs to target tumors located in the abdominal space, taking advantage of the low diffusion rate of benzimidazoles into the circulation and therefore maintaining high concentration at the tumor site.

While this may be advantageous for oral or regional treatment in order to accumulate drug in the GI tract, effective anti-metastasis treatment may require formulations that deliver larger systemic loads to the plasma. Many conventional benzimidazole formulations are designed to target intestinal parasites or topical fungal infections where high solubility is not a priority. Consequently, these formulations may limit this systemic application for disseminated tumors. One of ordinary skill in the art can determine whether such formulations are suitable for systemic administration of drug.

Using an improvised vehicle consisting of organic solvents and non-ionic surfactants, it has been demonstrated that benzimidazoles improved the survival of mice with metastatic lesions in the lung. Increased systemic bioavailability was confirmed by measurement of plasma levels of benzimidazole metabolites. When prepared in micelle solution, these drugs exhibit greater anti-tumor effects in the animals when compared to the original vehicle (DMSO).

B. Other Active Agents

The composition can further contain one or more additional active agents, such as diagnostic agents, therapeutic agents, and/or prophylactic agents. Suitable classes of active agents include, but are not limited to:

Cytotoxic anticancer agents, such as paclitaxel;

Cytostatic and/or cytotoxic agents such as anti-angiogenic agents such as avastin, endostatin, angiostatin, thalidomide, and revlimid;

Analgesics, such as opioid and non-opioid analgesics; and

Vaccines containing cancer antigens or immunomodulators, such as cytokines, to enhance the anti-cancer activity.

In one embodiment, the other active agent is not pentamidine or a metabolite thereof.

The benzimidazole and the one or more additional active agents can be formulated in the same dosage unit or separate dosage units. The benzimidazole and the one or more additional active agents can be administered simultaneously in the same dosage unit or separate dosage units or can be administered sequentially, for example, administration of the benzimidazole followed by administration of the one or more additional active agents or vice versa. If the benzimidazole and the one or more active agents are administered sequentially, the second agent to be administered is administered less than 4 hours following administration of the first agent, preferably less than 2 hours after the first agent, more preferably less than one hour after the first agent, more preferably less than 30 minutes after the first agent, most preferably immediately after administration of the first agent. “Immediately”, as used here, means less than 10 minutes, preferably less than 5 minutes, more preferably less than 2 minutes, most preferably less than one minute.

If in separate dosage units, the benzimidazole and the one or more additional active agents can be administered by the same route of administration or by different route of administration. For example, the benzimidazole and the one or more additional active agents can both be administered parenterally, or one can be administered parenterally and one orally.

### III. Formulations

The benzimidazoles described herein have typically been used to treat parasitic infections in the gastrointestinal tract, specifically in the intestine, and therefore are formulated to reside in the intestine, not for systemic uptake. Thus, these formulations are not optimal for systemic administration, for example, for parenteral administration, in part due to extreme insolubility and hydrophobicity. Dosages for treatment of intestinal worms and for treatment of metastatic cancer are also quite different.

Formulations have been developed for administration of other highly insoluble and/or hydrophobic chemotherapeutics such as taxol. For example, cremophor can be used to solubilize drugs such as taxol for intravenous administration. In another embodiment, the taxol is formulated with surfactant and spray dried as microparticles which are resuspended and dissolve following administration. In another embodiment, the chemotherapeutic is administered from an implanted pump

In a preferred embodiment, the one or more benzimidazoles are formulated as an emulsion for parenteral administration in a carrier containing a dipolar aprotic solvent, a highly polar solvent, a non-ionic surfactant, and an emulsifier. In a preferred embodiment, the formulation is in the form of a microemulsion. Microemulsions are generally clear, stable, isotropic liquid mixtures of oil, water or other polar solvent, and surfactant, frequently in combination with a co-surfactant. The droplets typically have a size less than 100 nm. The resulting mixture is stable and can be stored in room temperature. Physically stable refers to a microemulsion that remains in suspension, without precipitation, for one to three days. The mixture can be diluted in saline prior to administration.

Suitable dipolar aprotic solvents include, but are not limited to, N,N-dimethylacetamide (DMA), dimethylsulfoxide (DMSO), and combinations thereof. Suitable highly polar solvents include, but are not limited to, N-methyl-2-pyrrolidone.

Suitable non-ionic surfactants include, but are not limited to, Tweens, Arelacels, Transcutol, Capmul MCM, ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamine, polyoxyethylene hydrogenated tallow amide, or combinations thereof.

Suitable pharmaceutically acceptable emulsifiers include, but are not limited to polyethoxylated castor oils, such as Cremophor (e.g., Cremophor EL); carbohydrate materials, such as acacia, traganth, agar, chondrus, and pectin; proteins, such as gelatin, lecithin, and casein; high molecular weight alcohols, such as stearyl alcohol, cetyl alcohol, glyceryl monostearate, cholesterol and cholesterol stearates; wetting agents (e.g., anionic, cationic, or non-ionic), such as monovalent, polyvalent, or organic soaps (e.g., triethanol amine oleate, sulfonates, such as sodium lauryl sulfate, and benzalkonium chloride); and finely divided solids, such as colloidal clays.

The aprotic solvent, highly polar solvent, non-ionic surfactant, and emulsifier can be present in any amount. In one embodiment, the concentration of the aprotic solvent is from about 5% to about 20% by weight of the carrier, preferably from about 5% to about 15% by weight of the carrier, more preferably from about 10% to about 15% by weight of the carrier; the concentration of the highly polar solvent is from about 25% to about 50% by weight of the carrier, preferably from 30% to about 40% by weight of the carrier, more preferably from about 35% to about 40% by weight of the carrier; the concentration of the non-ionic surfactant is from about 15% to about 40% by weight of the carrier, preferably from about 20% to about 30% by weight of the carrier, more preferably from about 25% to about 30% by weight of the carrier; and the concentration of the emulsifier is from about 15% to about 40% by weight of the carrier, preferably from about 20% to about 30% by weight of the carrier, more preferably from about 25% to about 30% by weight of the carrier.

In the embodiment used in the examples, the carrier contains dimethylsulfoxide (12.5% by weight of the carrier), N-methyl-2-pyrrolidone (NMP, 37.5% by weight of the carrier), TWEEN®-80 (25% by weight of the carrier), and Cremophor EL (25% by weight of the carrier), herein referred to as DNTC, at a ratio of 1:3:2:2. The microemulsion suspensions are stable at room temperature for about one to three days.

In one embodiment, the concentration of the benzimidazole in DTNC is from about 0.01 mg/ml to 10 mg/ml, preferably from 0.01 mg/ml to 5 mg/ml, more preferably from 0.01 mg/ml to 3 mg/ml, most preferably from 0.01 mg/ml to 1 mg/ml. In another embodiment, the concentration of the benzimidazole in DTNC is from about 0.1 mg/ml to 10 mg/ml, preferably from 0.1 mg/ml to 5 mg/ml, more preferably from 0.1 mg/ml to 3 mg/ml, most preferably from 0.1 mg/ml to 1 mg/ml, most preferably from 0.1 mg/ml to 0.5 mg/ml. In still another embodiment, the concentration is greater than 1.0 mg/ml, for example, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, or 3.5 mg/ml. In still another embodiment, the concentration is from 0.3 to 3.0 mg/ml, for example, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mg/ml.

This vehicle did not cause significant toxicity to cultured cells. In addition, fenbendazole solubilized in DNTC showed increased cytotoxicity for prostate cancer cells compared to those solubilized in DMSO with a ˜2-fold reduction in ED50 (defined as the dose that kills 50% of the cell population) when tested in two human prostate cancer cell lines, PC-3M and PC-3MLN4.

To further evaluate the use of the DNTC as a vehicle in vivo, the bioavailability of this formulation was determined by measuring the plasma level of fenbendazole and its metabolites, fenbendazole sulfone and sulfoxide. Mice were injected with a bolus dose of fenbendazole, formulated either in DNTC or DMSO, at 50 and 150 mg/kg. At 8 hours post injection, plasma samples were collected and subjected to high performance liquid chromatography (HPLC) analysis. At both 50 and 150 mg/kg doses, the DNTC formulation provided greater than 10 fold increase in the level of fenbendazole metabolites detected in the plasma, when compared to the DMSO formulation, indicative of increased systemic drug absorption. Similar results were found with a second benzimidazole compound, albendazole when formulated in DNTC. Increased bioavailability resulted in a significant advantage in the treatment of animals bearing prostate cancer colonies in the lung. The use of surfactants may act to prolong the exposure and thus efficacy of these drugs These data strongly suggest that the DNTC vehicle improves the formulation of benzimidazoles for in vivo application while maintaining the cytotoxic effects on prostate cancer cells.

Parenteral and oral formulations of benzimidazoles, in the form of nanosuspensions, are described in U.S. Patent Application Publication No. 2008/0293796 to Chow et al. (“Chow”), which is incorporated herein by reference where appropriate. The nanosuspension may contain one or more surface polyols, dipolar aprotic solvents, stabilizers, and/or surfactants, and optionally water.

Suitable polyols include, but are not limited to, polyethylene glycol (PEG) 200, PEG 300, PEG 400, PEG 600, 1,2-propylene diol, glycerol, ethylene glycol, and combinations thereof.

A dipolar aprotic solvent is a solvent with a comparatively high relative dielectric constant, e.g., greater than about 15, and a sizable permanent dipole moment, that cannot donate suitably labile hydrogen atoms to form strong hydrogen bonds. One of ordinary skill in the art would be familiar with the class of agents typically known as dipolar aprotic solvents, and would understand that any of these agents may be included in the pharmaceutical compositions described herein, provided the solvent or solvents do not cause significant adverse reactions. Suitable dipolar aprotic solvents include, but are not limited to, N,N-dimethylacetamide (DMA), dimethylsulfoxide (DMSO), and combinations thereof.

The pharmaceutical compositions described herein may contain any ratio of polyol to dipolar aprotic solvent by weight. However, in certain embodiments, the ratio of polyol to dipolar aprotic solvent by weight is about 3:1. For example, the pharmaceutical compositions may include PEG 400 and N,N-dimethylacetamide in a ratio of 3:1 by weight. Alternatively, certain embodiments may include PEG 400 and dimethylsulfoxide in a 3:1 ratio by weight.

In some embodiments, the ratio of polyol to dipolar aprotic solvent is 7:1 by weight. For example, the pharmaceutical composition may contain PEG 400 and dimethylsulfoxide in a ratio of 7:1 by weight, or PEG 400 and N,N-dimethylacetamide in a ratio of 7:1 by weight.

In other embodiments, the composition includes water. Any amount of water may be used. However, in certain embodiments, the relative ratio of polyol:dipolar aprotic solvent:water is about 3:1:1 by weight. For example, the pharmaceutical compositions may contain PEG 400:N,N-dimethylacetamide:water in a 3:1:1 ratio by weight. Alternatively, other embodiments of the present pharmaceutical compositions include PEG 400:dimethylsulfoxide:water in a 3:1:1 ratio by weight.

Suitable surfactants include Tweens, Arelacels, and combinations thereof.

A. Controlled release formulations

The parenteral formulations described herein can be formulated for controlled release including immediate release, delayed release, extended release, pulsatile release, and combinations thereof.

### 1. Nano- and Micropartieles

For parenteral administration, the benzimidazole, and optional one or more additional active agents, can be incorporated into microparticles, nanoparticles, or combinations thereof that provide controlled release of the benzimidazole and/or one or more additional active agents. In embodiments wherein the formulations contains two or more drugs, the drugs can be formulated for the same type of controlled release (e.g., delayed, extended, immediate, or pulsatile) or the drugs can be independent formulated for a different type of release (e.g., immediate and delayed, immediate and extended, delayed and extended, delayed and pulsatile, etc.).

For example, the benzimidazole and/or one or more additional active agents can be incorporated into polymeric microparticles which provide controlled release of the drug(s). Release of the drug(s) is controlled by diffusion of the drug(s) out of the microparticles and/or degradation of the polymeric particles by hydrolysis and/or enzymatic degradation. Suitable polymers include ethylcellulose and other natural or synthetic cellulose derivatives. Polymers which are slowly soluble and form a gel in an aqueous environment, such as hydroxypropyl methylcellulose or polyethylene oxide may also be suitable as materials for drug containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the drug(s) can be incorporated into microparticles prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material which is normally solid at room temperature and has a melting point of from about 30 to 300° C.

In some cases, it may be desirable to alter the rate of water penetration into the microparticles. To this end, rate-controlling (wicking) agents may be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulose, and carboxymethyl-cellulose), alginic acid, lactose and talc. Additionally, a pharmaceutically acceptable surfactant (for example, lecithin) may be added to facilitate the degradation of such microparticles.

Proteins which are water insoluble, such as zein, can also be used as materials for the formation of drug containing microparticles. Additionally, proteins, polysaccharides and combinations thereof which are water soluble can be formulated with drug into microparticles and subsequently cross-linked to form an insoluble network. For example, cyclodextrins can be complexed with individual drug molecules and subsequently cross-linked.

Encapsulation or incorporation of drug into carrier materials to produce drug containing microparticles can be achieved through known pharmaceutical formulation techniques. In the case of formulation in fats, waxes or wax-like materials, the carrier material is typically heated above its melting temperature and the drug is added to form a mixture comprising drug particles suspended in the carrier material, drug dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, drug is added, and the molten wax-drug mixture is congealed under constant stirring as the mixture cools. Alternatively, the molten wax-drug mixture can be extruded and spheronized to form pellets or beads. Detailed descriptions of these processes can be found in “Remington—The science and practice of pharmacy”, 20th Edition, Jennaro et. al., (Phila, Lippencott, Williams, and Wilkens, 2000).

For some carrier materials it may be desirable to use a solvent evaporation technique to produce drug containing microparticles. In this case drug and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some embodiments, drug in a particulate form is homogeneously dispersed in a water-insoluble or slowly water soluble material. To minimize the size of the drug particles within the composition, the drug powder itself may be milled to generate fine particles prior to formulation. The process of jet milling, known in the pharmaceutical art, can be used for this purpose. In some embodiments drug in a particulate form is homogeneously dispersed in a wax or wax like substance by heating the wax or wax like substance above its melting point and adding the drug particles while stirring the mixture. In this case a pharmaceutically acceptable surfactant may be added to the mixture to facilitate the dispersion of the drug particles.

i. Coated Nano- and Microparticles

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin (Cortesi, R., et al., Biomaterials 19 (1998) 1641-1649). Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing microparticles or drug particles, a water soluble protein can be spray coated onto the microparticles and subsequently cross-linked by the one of the methods described above. Alternatively, drug containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

b. Embolytic Formulations

Embolizations (therapeutic vascular occlusions) are used to treat or prevent a range of pathological conditions in situ, including, for example, tumors, vascular malformations, and hemorrhagic processes. They can be performed in a variety of vessels or organs whether healthy or diseased. In these procedures, particulate occlusion agents (emboli) are positioned in the circulatory system using catheters under imagery control. U.S. Pat. No. 6,680,046 to Boschetti reports the following benefits of embolization. In the case of tumors, vascular occlusion can suppress pain, limit blood loss during surgical intervention following embolization or even bring on tumoral necrosis and avoid the necessity for surgical intervention. In the case of vascular malformations, embolization enables the blood flow to the “normal” tissues to be normalized, aids in surgery and limits the risk of hemorrhage. In hemorrhagic events or processes, vascular occlusion produces a reduction of blood flow, which promotes cicatrization of the arterial opening(s). Further, depending on the pathological conditions treated, embolization can be used for temporary as well as permanent objectives.

A range of solid materials, including polyvinyl alcohol (PVA) and polyacrylamide, have been used in embolization procedures. Several patents have also disclosed the combination of some of these materials with imaging and active agents, such as cell adhesion promoters. For example, U.S. Pat. No. 5,635,215 discloses microspheres comprising a hydrophilic acrylic copolymer coated with a cell adhesion promoter and a marking agent, which are useful for embolization. U.S. Pat. No. 5,648,100 discloses an injectable solution for therapeutic embolization, comprising microspheres comprising a hydrophilic acrylic copolymer coated with a cell adhesion promoter and a marking agent, and method of use.

c. Targeting to Tumors

The benzimidazoles can be bound to, or encapsulated within particles having on their surface, molecules that bind to antigens, ligands or receptors that are specific to tumor cells or tumor-associated neovasculature, or are upregulated in tumor cells or tumor-associated neovasculature compared to normal tissue, in order to target the drugs to the tumors.

### 1. Antigens, Ligands and Receptors to Target

a. Tumor-Specific and Tumor-Associated Antigens

The antigen expressed by the tumor may be specific to the tumor, or may be expressed at a higher level on the tumor cells as compared to non-tumor cells. Antigenic markers such as serologically defined markers known as tumor associated antigens, which are either uniquely expressed by cancer cells or are present at markedly higher levels (e.g., elevated in a statistically significant manner) in subjects having a malignant condition relative to appropriate controls, are contemplated for use in certain embodiments.

Tumor-associated antigens may include, for example, cellular oncogene-encoded products or aberrantly expressed proto-oncogene-encoded products (e.g., products encoded by the neu, ras, trk, and kit genes), or mutated forms of growth factor receptor or receptor-like cell surface molecules (e.g., surface receptor encoded by the c-erb B gene). Other tumor-associated antigens include molecules that may be directly involved in transformation events, or molecules that may not be directly involved in oncogenic transformation events but are expressed by tumor cells (e.g., carcinoembryonic antigen, CA-125, melanoma associated antigens, etc.) (see, e.g., U.S. Pat. No. 6,699,475; Jager, et al., Int. J. Cancer, 106:817-20 (2003); Kennedy, et al., Int. Rev. Immunol., 22:141-72 (2003); Scanlan, et al. Cancer Immun., 4:1 (2004)).

Genes that encode cellular tumor associated antigens include cellular oncogenes and proto-oncogenes that are aberrantly expressed. In general, cellular oncogenes encode products that are directly relevant to the transformation of the cell, and because of this, these antigens are particularly preferred targets for immunotherapy. An example is the tumorigenic neu gene that encodes a cell surface molecule involved in oncogenic transformation. Other examples include the ras, kit, and trk genes. The products of proto-oncogenes (the normal genes which are mutated to form oncogenes) may be aberrantly expressed (e.g., overexpressed), and this aberrant expression can be related to cellular transformation. Thus, the product encoded by proto-oncogenes can be targeted. Some oncogenes encode growth factor receptor molecules or growth factor receptor-like molecules that are expressed on the tumor cell surface. An example is the cell surface receptor encoded by the c-erbB gene. Other tumor-associated antigens may or may not be directly involved in malignant transformation. These antigens, however, are expressed by certain tumor cells and may therefore provide effective targets. Some examples are carcinoembryonic antigen (CEA), CA 125 (associated with ovarian carcinoma), and melanoma specific antigens.

In ovarian and other carcinomas, for example, tumor associated antigens are detectable in samples of readily obtained biological fluids such as serum or mucosal secretions. One such marker is CA125, a carcinoma associated antigen that is also shed into the bloodstream, where it is detectable in serum (e.g., Bast, et al., N. Eng. J. Med., 309:883 (1983); Lloyd, et al., Int. J. Canc., 71:842 (1997). CA125 levels in serum and other biological fluids have been measured along with levels of other markers, for example, carcinoembryonic antigen (CEA), squamous cell carcinoma antigen (SCC), tissue polypeptide specific antigen (TPS), sialyl TN mucin (STN), and placental alkaline phosphatase (PLAP), in efforts to provide diagnostic and/or prognostic profiles of ovarian and other carcinomas (e.g., Sarandakou, et al., Acta Oncol., 36:755 (1997); Sarandakou, et al., Eur. J. GynaecoL Oncol., 19:73 (1998); Meier, et al., Anticancer Res., 17(4B):2945 (1997); Kudoh, et al., Gynecol. Obstet. Invest., 47:52 (1999)). Elevated serum CA125 may also accompany neuroblastoma (e.g., Hirokawa, et al., Surg. Today, 28:349 (1998), while elevated CEA and SCC, among others, may accompany colorectal cancer (Gebauer, et al., Anticancer Res., 17(4B):2939 (1997)).

The tumor associated antigen, mesothelin, defined by reactivity with monoclonal antibody K-1, is present on a majority of squamous cell carcinomas including epithelial ovarian, cervical, and esophageal tumors, and on mesotheliomas (Chang, et al., Cancer Res., 52:181 (1992); Chang, et al., Int. J. Cancer, 50:373 (1992); Chang, et al., Int. J. Cancer, 51:548 (1992); Chang, et al., Proc. Natl. Acad. Sci. USA, 93:136 (1996); Chowdhury, et al., Proc. Natl. Acad. Sci. USA, 95:669 (1998)). Using MAb K-1, mesothelin is detectable only as a cell-associated tumor marker and has not been found in soluble form in serum from ovarian cancer patients, or in medium conditioned by OVCAR-3 cells (Chang, et al., Int. J. Cancer, 50:373 (1992)). Structurally related human mesothelin polypeptides, however, also include tumor-associated antigen polypeptides such as the distinct mesothelin related antigen (MRA) polypeptide, which is detectable as a naturally occurring soluble antigen in biological fluids from patients having malignancies (see WO 00/50900).

A tumor antigen may be a cell surface molecule. Tumor antigens of known structure and having a known or described function, include the following cell surface receptors: HER1 (GenBank Accession No. U48722), HER2 (Yoshino, et al., J. Immunol., 152:2393 (1994); Disis, et al., Canc. Res., 54:16 (1994); GenBank Acc. Nos. X03363 and M17730), HER3 (GenBank Ace. Nos. U29339 and M34309), HER4 (Plowman, et al., Nature, 366:473 (1993); GenBank Ace. Nos. L07868 and T64105), epidermal growth factor receptor (EGFR) (GenBank Acc. Nos. U48722, and KO30193), vascular endothelial cell growth factor (GenBank No. M32977), vascular endothelial cell growth factor receptor (GenBank Acc. Nos. AF022375, 1680143, U48801 and X62568), insulin-like growth factor-1 (GenBank Ace. Nos. X00173, X56774, X56773, X06043, European Patent No. GB 2241703), insulin-like growth factor-II (GenBank Acc. Nos. X03562, X00910, M17863 and M17862), transferrin receptor (Trowbridge and Omary, Proc. Nat. Acad USA, 78:3039 (1981); GenBank Ace. Nos. X01060 and M11507), estrogen receptor (GenBank Ace. Nos. M38651, X03635, X99101, U47678 and M12674), progesterone receptor (GenBank Acc. Nos. X51730, X69068 and M15716), follicle stimulating hormone receptor (FSH—R) (GenBank Acc. Nos. Z34260 and M65085), retinoic acid receptor (GenBank Ace. Nos. L12060, M60909, X77664, X57280, X07282 and X06538), MUC-1 (Barnes, et al., Proc. Nat. Acad. Sci. USA, 86:7159 (1989); GenBank Aec. Nos. M65132 and M64928) NY-ESO-1 (GenBank Acc. Nos. AJ003149 and U87459), NA 17-A (PCT Publication No. WO 96/40039), Melan-A/MART-1 (Kawakami, et al., Proc. Nat. Acad. Sci. USA, 91:3515 (1994); GenBank Ace. Nos. U06654 and U06452), tyrosinase (Topalian, et al., Proc. Nat. Acad. Sci. USA, 91:9461 (1994); GenBank Ace. No. M26729; Weber, et al., J. Clin. Invest, 102:1258 (1998)), Gp-100 (Kawakami, et al., Proc. Nat. Acad. Sci. USA, 91:3515 (1994); GenBank Am. No. 573003, Adema, et al., J. Biol. Chem., 269:20126 (1994)), MAGE (van den Bruggen, et al., Science, 254:1643 (1991)); GenBank Ace. Nos. U93163, AF064589, U66083, D32077, D32076, D32075, U10694, U10693, U10691, U10690, U10689, U10688, U10687, U10686, U10685, L18877, U10340, U10339, L18920, UO3735 and M77481), BAGE (GenBank Acc. No. U19180; U.S. Pat. Nos. 5,683,886 and 5,571,711), GAGE (GenBank Ace. Nos. AF055475, AF055474, AF055473, U19147, U19146, U19145, U19144, U19143 and U19142), any of the CTA class of receptors including in particular HOM-MEL-40 antigen encoded by the SSX2 gene (GenBank Ace. Nos. X86175, U90842, U90841 and X86174), carcinoembryonic antigen (CEA, Gold and Freedman, J. Exp. Med., 121:439 (1985); GenBank Ace. Nos. M59710, M59255 and M29540), and PyLT (GenBank Ace. Nos. J02289 and J02038); p97 (melanotransferrin) (Brown, et al., J. Immunol., 127:539-46 (1981); Rose, et al., Proc. Natl. Acad. Sci. USA, 83:1261-61 (1986)).

Additional tumor associated antigens include prostate surface antigen (PSA) (U.S. Pat. Nos. 6,677,157; 6,673,545); β-human chorionic gonadotropin 13-HCG) (McManus, et al., Cancer Res., 36:3476-81 (1976); Yoshimura, et al., Cancer, 73:2745-52 (1994); Yamaguchi, et al., Br. J. Cancer, 60:382-84 (1989): Alfthan, et al., Cancer Res., 52:4628-33 (1992)); glycosyltransferase β-1,4-N-acetylgalactosaminyltransferases (GalNAc) (Hoon, et al., Int. J. Cancer, 43:857-62 (1989); Ando, et al., Int. J. Cancer, 40:12-17 (1987); Tsuchida, et al., J. Natl. Cancer, 78:45-54 (1987); Tsuchida, et al., J. Natl. Cancer, 78:55-60 (1987)); NUC18 (Lehmann, et al., Proc. Natl. Acad. Sci. USA, 86:9891-95 (1989); Lehmann, et al., Cancer Res., 47:841-45 (1987)); melanoma antigen gp75 (Vijayasardahi, et al., J. Exp. Med., 171:1375-80 (1990); GenBank Accession No. X51455); human cytokeratin 8; high molecular weight melanoma antigen (Natali, et al., Cancer, 59:55-63 (1987); keratin 19 (Datta, et al., J. Clin. Oncol., 12:475-82 (1994)).

Tumor antigens of interest include antigens regarded in the art as “cancer/testis” (CT) antigens that are immunogenic in subjects having a malignant condition (Scanlan, et al., Cancer Immun., 4:1 (2004)). CT antigens include at least 19 different families of antigens that contain one or more members and that are capable of inducing an immune response, including but not limited to MAGEA (CT1); BAGE (CT2); MAGEB (CT3); GAGE (CT4); SSX (CT5); NY-ESO-1 (CT6); MAGEC (CT7); SYCP1 (C8); SPANXB1 (CT11.2); NA88 (CT18); CTAGE (CT21); SPA17 (CT22); OY-TES-1 (CT23); CAGE (CT26); HOM-TES-85 (CT28); HCA661 (CT30); NY-SAR-35 (CT38); FATE (CT43); and TPTE (CT44).

Additional tumor antigens that can be targeted, including a tumor-associated or tumor-specific antigen, include, but not limited to, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pml-RARαfusion protein, PTPRK, K-ras, N-ras, Triosephosphate isomeras, Bage-1, Gage 3,4,5,6,7, GnTV, Herv-K-mel, Lage-1, Mage-A1,2,3,4,6,10,12, Mage-C2, NA-88, NY-Eso-1/Lage-2, SP17, SSX-2, and TRP2-Int2, MelanA (MART-I), gp100 (Pinel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/MeI-40, PRAMS, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, α-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90 (Mac-2 binding protein\cyclophilin C-associated protein), TAAL6, TAG72, TLP, and TPS. Other tumor-associated and tumor-specific antigens are known to those of skill in the art and are suitable for targeting by the disclosed benzimidazoles.

b. Antigens Associated with Tumor Neovasculature

Tumor-associated neovasculature provides a readily accessible route through which benzimidazoles can access the tumor. In another embodiment the benzimidazoles contain a domain that specifically binds to an antigen that is expressed by neovasculature associated with a tumor.

The antigen may be specific to tumor neovasculature or may be expressed at a higher level in tumor neovasculature when compared to normal vasculature. Exemplary antigens that are over-expressed by tumor-associated neovasculature as compared to normal vasculature include, but are not limited to, VEGF/KDR, Tie2, vascular cell adhesion molecule (VCAM), endoglin and α₅β₃ integrin/vitronectin. Other antigens that are over-expressed by tumor-associated neovasculature as compared to normal vasculature are known to those of skill in the art and are suitable for targeting by the disclosed benzimidazoles.

### 2. Molecular Classes of Targeting Domains

a. Ligands and Receptors

In one embodiment, tumor or tumor-associated neovasculature targeting domains are ligands that bind to cell surface antigens or receptors that are specifically expressed on tumor cells or tumor-associated neovasculature or are overexpressed on tumor cells or tumor-associated neovasculature as compared to normal tissue. Tumors also secrete a large number of ligands into the tumor microenvironment that affect tumor growth and development. Receptors that bind to ligands secreted by tumors, including, but not limited to growth factors, cytokines and chemokines, including the chemokines provided above, are suitable for use in the disclosed benzimidazoles. Ligands secreted by tumors can be targeted using soluble fragments of receptors that bind to the secreted ligands. Soluble receptor fragments are fragments polypeptides that may be shed, secreted or otherwise extracted from the producing cells and include the entire extracellular domain, or fragments thereof.

b. Single Polypeptide Antibodies

In another embodiment, tumor or tumor-associated neovasculature targeting domains are single polypeptide antibodies that bind to cell surface antigens or receptors that are specifically expressed on tumor cells or tumor-associated neovasculature or are overexpressed on tumor cells or tumor-associated neovasculature as compared to normal tissue.

c. Fc Domains

In another embodiment, tumor or tumor-associated neovasculature targeting domains are Fc domains of immunoglobulin heavy chains that bind to Fc receptors expressed on tumor cells or on tumor-associated neovasculature. The Fc region as used herein includes the polypeptides containing the constant region of an antibody excluding the first constant region immunoglobulin domain. Thus Fc refers to the last two constant region immunoglobulin domains of IgA, IgD, and IgG, and the last three constant region immunoglobulin domains of IgE and IgM. In a preferred embodiment, the Fc domain is derived from a human or murine immunoglobulin. In a more preferred embodiment, the Fc domain is derived from human IgG1 or murine IgG2a including the C_{H}2 and C_{H}3 regions.

c. Glycophosphatidylinositol Anchor Domain

In another embodiment, tumor or tumor-associated neovasculature targeting domains are polypeptides that provide a signal for the posttranslational addition of a glycosylphosphatidylinositol (GPI) anchor. GPI anchors are glycolipid structures that are added posttranslationally to the C-terminus of many eukaryotic proteins. This modification anchors the attached protein in the outer leaflet of cell membranes. GPI anchors can be used to attach T cell receptor binding domains to the surface of cells for presentation to T cells. In this embodiment, the GPI anchor domain is C-terminal to the T cell receptor binding domain.

In one embodiment, the GPI anchor domain is a polypeptide that signals for the posttranslational addition of a GPI anchor when the polypeptide is expressed in a eukaryotic system. Anchor addition is determined by the GPI anchor signal sequence, which consists of a set of small amino acids at the site of anchor addition followed by a hydrophilic spacer and ending in a hydrophobic stretch (Low, FASEB J., 3:1600-1608 (1989)). Cleavage of this signal sequence occurs in the ER before the addition of an anchor with conserved central components (Low, FASEB J., 3:1600-1608 (1989)) but with variable peripheral moieties (Homans et al., Nature, 333:269-272 (1988)). The C-terminus of a GM-anchored protein is linked through a phosphoethanolamine bridge to the highly conserved core glycan, mannose (α1-2) mannose (α1-6) mannose (α1-4) glucosamine (α1-6) myoinositol.

A phospholipid tail attaches the GPI anchor to the cell membrane. The glycan core can be variously modified with side chains, such as a phosphoethanolamine group, mannose, galactose, sialic acid, or other sugars. The most common side chain attached to the first mannose residue is another mannose. Complex side chains, such as the N-acetylgalactosamine-containing polysaccharides attached to the third mannose of the glycan core, are found in mammalian anchor structures. The core glucosamine is rarely modified. Depending on the protein and species of origin, the lipid anchor of the phophoinositol ring is a diacylglycerol, an alkylacylglycerol, or a ceramide. The lipid species vary in length, ranging from 14 to 28 carbons, and can be either saturated or unsaturated. Many GPI anchors also contain an additional fatty acid, such as palmitic acid, on the 2-hydroxyl of the inositol ring. This extra fatty acid renders the GPI anchor resistant to cleavage by PI-PLC.

GPI anchor attachment can be achieved by expression of a fusion protein containing a GPI anchor domain in a eukaryotic system capable of carrying out GPI posttranslational modifications. GPI anchor domains can be used as the tumor or tumor vasculature targeting domain, or can be additionally added to benzamidizoles already containing separate tumor or tumor vasculature targeting domains.

In another embodiment, GPI anchor moieties are added directly to isolated T cell receptor binding domains through an in vitro enzymatic or chemical process. In this embodiment, GPI anchors can be added to polypeptides without the requirement for a GPI anchor domain. Thus, GPI anchor moieties can be added to benzamidizoles described herein having a T cell receptor binding domain and a tumor or tumor vasculature targeting domain. Alternatively, GPI anchors can be added directly to T cell receptor binding domain polypeptides without the requirement for fusion partners encoding tumor or tumor vasculature targeting domains.

### IV. Methods of Use

The compositions and formulations described herein can be used to treat metastatic cancer. In one embodiment, a patient can be screened for the presence of metastatic tumors and treated with a composition containing an effective amount of one or more active agents to treat the metastatic tumors.

In one embodiment, the formulations described herein are used to treat disseminated, hormone-refractory prostate cancer which, for example, has metastasized to the lungs, bone, or combinations thereof. Hormone-refractory prostate cancer generally refers to advanced prostate cancer characterized by three consecutive increases in prostate specific antigen (PSA) levels while the individual is still on hormone therapy. In one embodiment, the cancer is characterized by three consecutive increases in PSA levels of at least 10% each or three increases that involve an increase of 50% over the nadir PSA or an increase in tumor mass on bone scan, X-ray, CT scan or MRI despite a castrate level of testosterone (T<20 ng/dl). The therapeutic formulations are administered in a formulation, dosage, schedule and route of administration, for a period of time, as determined by one of skill in the art in treating cancer patients.

Typical dosing is from 0.1 to 500 mg benzimidazole/kg/day, preferably from 0.1 to 400 mg/kg/day, preferably from 0.1 to 300 mg/kg/day, more preferably from 0.1 to 250 mg/kg/day, more preferably from 0.1 to 200 mg/kg/day, most preferably from 0.1 to 150 mg/kg/day, 0.1 to 100 mg/kg/day, 0.1 to 50 mg/kg/day, or 0.1 to 25 mg/kg/day. In one embodiment, the dosage is from 5 to 1000 mg/day, preferably from 5 to 500 mg/day, preferably from 5 to 250 mg/day, preferably from 5 to 150 mg/day, more preferably from 5 to 100 mg/day, more preferably from 5 to 100 mg/day, most preferably from 5 to 50 mg/day. For example, the daily dose can be 5, 10, 15, 20, 25, 30, 35, 40, 45, 50, 55, 60, 65, 70, 75, 80, 85, 90, 95, or 100 mg/day or greater. In another embodiment, the daily dose is 110, 120, 130, 140, 150, 160, 170, 180, 190, 200, 210, 220, 230, 240, or 250 mg/day or greater. Dosage may be adjusted for delivery formulation, dosing regime, and combination therapy.

The formulations can be administered once day or more than once a day, such as twice a day, three times a day, or more or less frequently, such as once or more a week, for example, twice a week, three times a week, four times a week, or more. If the formulation is a controlled release formulation, it may be administered less frequently, such as once every two weeks, once every three weeks, or once a month.

Prostate cancer is known to metastasize to the lungs, bone, particularly the pelvis, spine, and ribs, and/or the lymph nodes. The examples below show that the formulations described herein are more cytotoxic against prostate cancer cell lines prone to metastasis, such as PC3MLN4 cells, than prostate cancer cell lines less prone to metastasis, such as PC3M cells.

Compared to PC-3M, PC-3MLN4 cells are highly metastatic to lymph nodes when implanted orthotopically in mouse prostate. DU-145LN4 and DU-145ivLU4 cells are developed from lymph node metastases after orthotopic injection and lung metastases after intravenous injection of DU-145 human prostate cancer in the mouse, respectively. These prostate cancer cells are androgen receptor negative as is frequently the case in hormone refractory prostate cancer disease. About 2% of the drug library was identified to possess selective cytotoxicity on metastatic variants over the parental cell lines, rather than general cytotoxic effects.

Benzimidazoles Show Greater Cytotoxicity in Highly Metastatic Cell Lines

Fenbendazole, albendazole, mebendazole, flubendazole and oxibendazole demonstrated greater cytotoxicity in the highly metastatic PC-3MLN4 and AT6.1 cells relative to the poorly metastatic PC-3M cells. These cytotoxic effects are mediated in part by an induction of mitotic arrest, as shown using histone H3 phospho-Serine-28 as a marker of G2/M arrest. The mitotic index for each agent was normalized against those obtained from vehicle-treated cells, and compared with the activity of paclitaxel, a known inducer of mitotic arrest. In all three prostate cancer cells tested, these five benzimidazoles showed potent induction of mitotic arrest (value greater than 1.00), with induction in the metastatic PC-3MLN4 cells. Interestingly, the potency of mitotic arrest induced by these compounds does not correlate directly with the degree of cell growth inhibition. For instance, treatment of PC-3MLN4 cells with 1 μM fenbendazole (ED50 0.9 μM) resulted in the highest induction of mitotic arrest (mitotic index 2.27); however, treatment with albendazole which has a lower ED50 (0.58 μM) and oxibendazole which has a higher ED50 (2.04 μM) exhibited mitotic index in the range of 1.8-2.0.

Using an in vitro tubulin polymerization assay, it was confirmed that benzimidazoles disrupt microtubule polymerization. Interestingly, benomyl, a benzimidazole compound that showed relatively low cytoxic activity with prostate cancer cells, exhibited the strongest tubulin disrupting effect. These results suggest that mechanisms other than microtubule disruption may have a role in the anti-tumor cell activity of the active benzimidazoles.

Benzimidazoles Preferentially Induce Apoptosis in Metastatic Cells

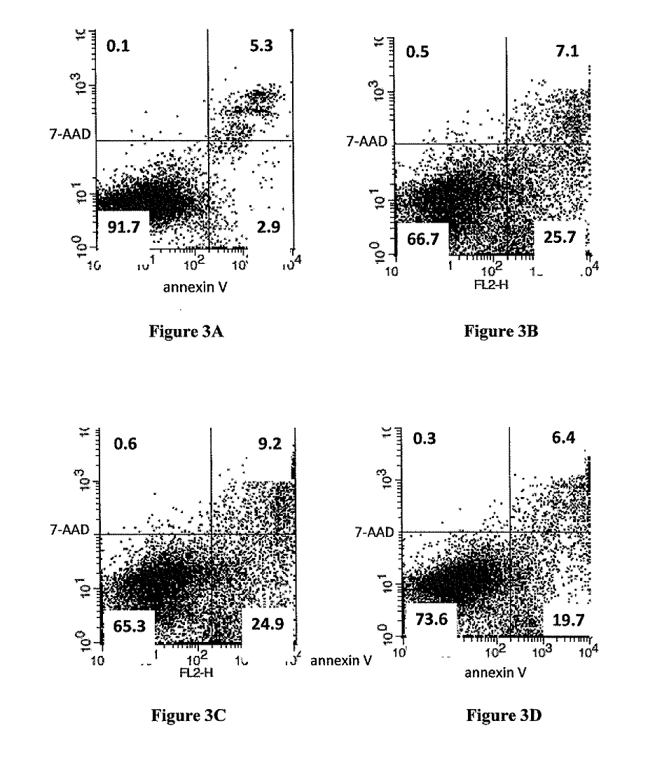

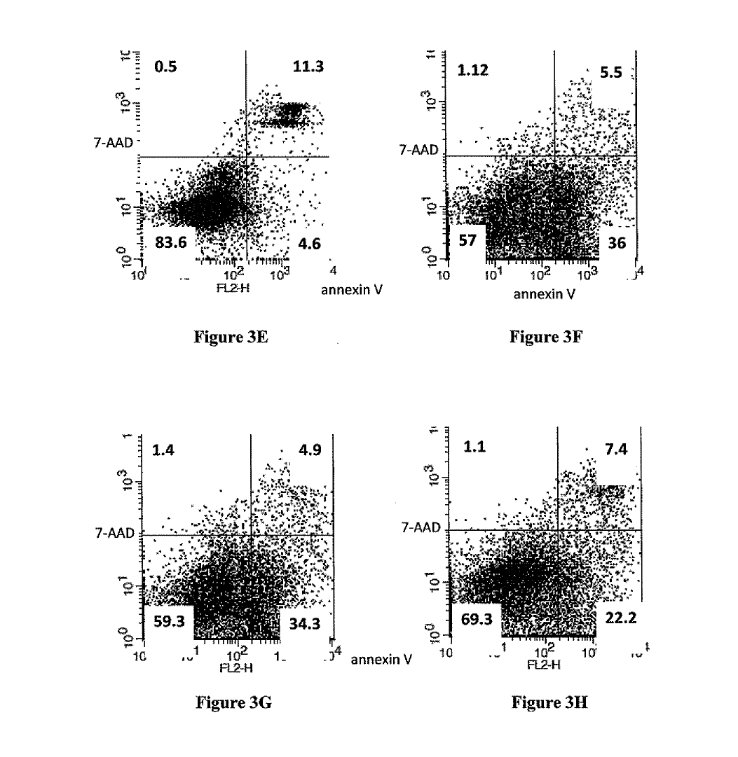

Benzimidazoles were evaluated for the ability to preferentially induce apoptosis in metastatic prostate cancer cells. Cells were treated with the drugs for 72 hours before staining for annexin V, an indicator of both early and late apoptosis. Treatment with benzimidazoles that displayed potent growth inhibitory effects earlier also resulted in a greater induction of apoptosis in PC-3MLN4 than in PC-3M cells. These effects were significantly reduced when PC-3MLN4 cells were concurrently treated with a caspase-3 inhibitor (z-VAD-FMK) (FIG. 3D). Taken together, fenbendazole, albendazole, mebendazole, flubendazole and oxibendazole all demonstrate cytotoxic activity against highly metastatic prostate cancer cells in vitro via induction of apoptosis.

Many anti-tumor drugs are tested only for their ability to prevent primary tumor growth or to prevent the dissemination of metastatic cells. To accomplish this, the drug is usually administered shortly after the primary tumor is established. To effectively treat metastatic cancer, a drug must have the ability to retard the growth of pre-established metastatic colonies. The benzimidazoles described herein were evaluated in an experimental metastases model in the lungs of nude mice following tail-vein injection of Dunning rat AT6.1 prostate carcinoma. Drug treatment was begun only after colonies were visible in the lungs of test mice, generally at 5 days post-inoculation. Mice were given intraperitoneal injections of vehicle, fenbendazole, albendazole or mebendazole (100 mg/kg) three times a week until signs of morbidity were observed. In order to achieve greater bioavailability, we first solubilized benzimidazoles in DNTC formulation and then diluted with saline prior to injection.

Animals treated with these drug preparations did not show overt signs of increased toxicity, as measured by loss of body weight or changes in animal appearance and behavior. Compared to the vehicle-treated group, mice receiving fenbendazole, albendazole or mebendazole showed significantly increased survival times. To distinguish differences in survival between these groups, we calculated the percent increase in life span (ILS %), defined as the percent increase in the mean time to death of drug-treated animals over the corresponding time to death for vehicle-treated mice. The results show that benzimidazoles significantly increase post-metastatic animal viability, with ILS %: fenbendazole (45.3%), albendazole (59.7%) and mebendazole (48.9%). These data demonstrate that multiple members of the benzimidazole family have potent activity in inhibiting the growth of disseminated prostate cancer and in extending the survival of mice bearing these tumors.

To further compare the mechanism of actions of benzimidazole compounds in vivo, Dunning rat AT6.1 lung metastases from animals treated with vehicle, fenbendazole, albendazole or mebendazole (100 mg/kg) for two weeks were examined. Similar to the survival model, treatment was not begun until day 5 when micrometastases were confirmed growing in the lung. For this approach, the AT6.1 cells were engineered to express secreted Gaussia luciferase enzyme. Using measurement of luciferase activity from the peripheral blood as an indicator of overall tumor burden, a significant reduction of tumor burden in the lungs of benzimidazole-treated animals when compared to vehicle-treated mice was observed. After 22 days, the mice were sacrificed and the lungs examined for viable tumor colonies. These results confirmed a decrease in lung tumor burden in benzimidazole-treated animals.

To investigate whether benzimidazoles treatment affects cell proliferation and apoptosis in vivo, immunohistochemical analysis of the cell proliferation marker, Ki-67 and of the apoptotic marker, activated caspase-3 was performed on mouse lung sections from each group. A reduction of nuclear staining of Ki-67 (P<0.005) and an increase of cytoplasmic staining of activated caspase-3 (P<0.005) was observed in lungs collected from benzimidazole-treated animals compared to vehicle-treated animals. These results were confirmed in vitro using propidium iodide and annexin V staining to analyze DNA distribution and apoptosis, respectively. These anti-parasitic compounds induced G₂-M cell cycle arrest at 6 hours post treatment, which led to a significant increase of apoptotic cell population after 72 hours post treatment. Preliminary studies showed that the treatment may induce microtubule dynamic instability for its anti-tumor effects. These data provide evidence that benzimidazoles inhibit cell proliferation and induce apoptosis in metastatic prostate cancer cells in vivo as well as in vitro.

It was also observed that treatment with benzimidazoles induced expression of HERPUD1, ATF3 and DDIT3, all of which are implicated in ER stress signaling. Lower HERPUD1 mRNA level in human prostate tumors has been correlated with a higher incidence of metastases and over-expression of this gene induces apoptosis in prostate cancer cells. ATF3 is required for tumor suppressor KLF family member to induce apoptosis in prostate cancer cells. Benzimidazole treatment may depend on these proteins to induce apoptosis.

Benzimidazoles are More Effective than Paclitaxel for the Treatment of Metastatic Cancers

Paclitaxel is one of the standard chemotherapy regimens available for men with metastatic prostate cancer. In the in viva Dunning AT6.1 rat prostate carcinoma model described above, it was determined that paclitaxel at 10 mg/kg was the optimal dose in terms of providing the highest survival rate without causing severe toxicity (loss of body weight and neurological side effects) when given three times a week. Albendazole given at 100 and 250 mg/kg was compared with 10 mg/kg paclitaxel using the same treatment schedule. Paclitaxel and albendazole-treated mice showed increased survival relative to the vehicle-treated group. The increase provided by treatment with albendazole at 250 mg/kg (ILS % 64) was significantly greater than that seen in animals treated with paclitaxel at 10 mg/kg (ILS %30.2) (P<0.01). Confirming the in vitro data, benomyl showed little or no extension of lifespan of tumor-bearing animals in vivo (ILS % 7.5). Collectively, it is shown that multiple benzimidazole compounds, including fenbendazole, albendazole and mebendazole, exert potent therapeutic efficacy in vivo by prolonging the survival of animals bearing pre-existing metastases. Notably, these anti-tumor effects were comparable to if not more effective than paclitaxel, the current standard chemotherapeutic agent available in the clinic.

Benzimidazoles are Effect Against Paclitaxel-Resistant Metastatic Cancers

Most men with metastatic prostate lesions who fail hormone deprivation therapy undergo paclitaxel-based chemotherapy, which provides a mean survival increase of 2 months. This limited efficacy is due primarily to the development of taxane-resistance in these tumors. The generation of taxane resistance is multifactoral, resulting from events including tubulin mutations, bcl-2 overexpression and increased levels of the multidrug resistance protein (MDR). To evaluate whether benzimidazoles are effective against taxane-resistant cells, we treated two paclitaxel-resistant prostate cancer cells, PC-3TxR and DU-145TxR, with benzimidazoles. These cells, developed from PC-3 and DU-145 cells, respectively, were treated continuously in vitro with paclitaxel until significant resistance was developed. These cells responded to the cytotoxic effects of benzimidazoles in a dose dependent manner, but were refractory to paclitaxel treatment. In fact, benzimidazoles exhibited higher potency (lower ED50) in the paclitaxel-resistant cells than in the parental cells. A significant cytotoxic effect was also observed with benzimidazoles in MES-SA/Dx5 human uterine sarcoma cells which are resistant to both microtubule stabilizer and disrupter agents. The observed cytotoxic activity correlated with the percent apoptosis induced in these cells.

The anti-tumor activity of the benzimidazoles on paclitaxel-resistant cells was confirmed in vivo. PC-3TxR cells were inoculated subcutaneously in the flanks of the nude mice. When the tumors reached approximately 100 mm³ in size, the mice were treated with vehicle, paclitaxel (10 mg/kg), fenbendazole (100 mg/kg) or albendazole (100 mg/kg) three times a week for 3 weeks. These benzimidazoles were prepared in DNTC and diluted with saline prior to injection. Compared to the vehicle treated group, the growth of PC-3TxR tumors in the paclitaxel group was significantly greater, indicating these cells require paclitaxel for optimal growth. In contrast, tumors treated with either fenbendazole or albendazole were growth inhibited and remained at approximately 100-150 mm³ in size throughout the study. These data strongly suggest benzimidazoles are effective against paclitaxel-resistant prostate cancer cells in vitro and in vivo. Combination therapies of benzimidazole and paclitaxel showed enhanced cytotoxicity against metastatic prostate tumors. These results suggest that benzimidazoles may be useful in the treatment of prostate cancer as an adjunct or sequel to taxane treatment.

Further, the active metabolite (sulfoxide derivative) of albendazole and fenbendazole were not substrates to human breast cancer resistance protein (Bcrp1/ABCG2), multidrug resistant protein (MRP) 2 or P-glycoprotein (Pgp) (37). These may explain the observations that benzimidazoles exhibit strong cytotoxicity against multi-drug resistant cancer cells, as well as against cells resistant to either microtubule stabilizers or disrupters. This could be particularly useful in the treatment of patients with metastatic prostate cancer whose tumors have become resistant to docetaxel. More importantly, the dose range that showed potent anti-tumor effects in this studies (low microMolar in vitro and 100 mg/kg in vivo) are within the achievable pharmacological levels in humans. Because of their relatively benign safety profile, it should be possible to use benzimidazoles in the long term treatment of metastatic cancer disease, similar to its current use in treating parasitic diseases in humans.

Benzimidazoles are Effective at Reducing Tumor Burden and Prolonging the Survival Times in Animals Having Metastases in the Lungs and Bone

In vivo data described in the examples illustrates that treatment of animals having metastatic tumors in the lungs or bone significantly increases the survival times of the animals versus a control.

In the lung metastasis model, treatment was initiated 10 days after the animals were inoculated with tumor cells. At this time, the lungs were riddled with metastases. Formulations containing a benzimidazole were injected into the peritoneum three times per week. Half of the control mice died by day 27, only 17 days after the animals started receiving treatments. In contrast, half of the mice treated with fenbendazole were still alive on day 37 and one-third were still alive at day 45. Efficacy studies showed that fenbendazole is equivalent to taxol in prolonging the survival times of the mice.

Using the metastatic prostate cancer PC-3MLN4 cells that expressed luciferase, the anti-tumor effects of benzimidazoles in a model of experimental bone metastasis were investigated. Cells were inoculated directly into a single tibia of immunocompromised mice via intraosseous injection. When bone lesions were confirmed to be growing via bioluminescent imaging, mice were randomized and treated with vehicle or with 100 mg/kg albendazole for two weeks. Mice treated with albendazole showed reduced tumor growth compared to those treated with vehicle. There was a significant difference in average luciferase signal between the two groups over the course of treatment (P<0.05).

At the end of study, the anatomical and radiographic characteristics of the bone lesions were evaluated with X-ray and micro-computerized tomography (CT) imaging. Mice treated with vehicle showed extensive osteolysis due to the osteoclastic bone resorption activity of PC-3MLN4 cells. In contrast, the bone integrity was maintained in albendazole-treated mice. Immunohistochemical analysis of these bone lesions further showed a reduction of Ki-67 labeling index (P<0.005) and an increase of apoptotic index, based on activated caspase-3 staining (P<0.005) in the albendazole-treated group. These results demonstrated that the anti-tumor effects of benzimidazoles extend to tumors growing in intraosseuous spaces.

The examples also demonstrate the cytotoxicity of fenbendazole, albendazole or mebendazole against various other human cancer types including ACHN human renal cell carcinoma cells, U2OS human osteosarcoma cells, AsPC-1, BxPC-3 and Capan-2 human pancreatic adenocarcinoma cells, HT1080 human fibrosarcoma cells, MESSA human uterine sarcoma, MCF-7 human breast cancer cells, A549 human lung adenocarcinoma cells and H460 human non-small cell lung cancer cells.

### EXAMPLES

The present invention will be further understood by reference to the following non-limiting examples.

### Materials and Methods

Chemicals and Reagents

The following reagents were purchased from Sigma Aldrich: fenbendazole (F5396), albendazole (A4673), mebendazole (M2523), oxibendazole (32924), flubendazole (34091), benomyl (381586), carbendazim (45368), thiabendazole (45684) and thiophanate-methyl (45688), dimethyl sulfoxide (DMSO) (D5879), TWEEN®-80 (P4780), N-methyl-2-pyrrolidone (NMP) (328634) and Cremophor® EL (Cr-EL) (C5135). Paclitaxel was purchased from Cytoskeleton (TXD01).

Cell Lines

AT6.1 Dunning rat prostate carcinoma provided by J. Isaacs (Johns Hopkins University) were maintained as described in the literature. PC3M and PC3MLN4 human prostate cancer cells were provided by J. Fidler. PC3, PC3-TR, DU145 and DU145-TR human prostate cancer cell lines were provided by E. Keller. A549, MESSA and MESSA-Dx5 were obtained from ATCC, and MCF7 and MCF7-TR were obtained from A. M. Parissenti. All PC3 derivative cell lines were maintained in RPMI 1640 supplemented with 10% fetal bovine serum and 1% penicillin/streptomycin. DU145 and MCF7 cell lines were maintained in DMEM media supplemented with 10% fetal bovine serum and 1% penicillin/streptomycin, while A549 and MESSA cell lines were maintained in F12K media supplemented with 10% fetal bovine serum and 1% penicillin/streptomycin. Paclitaxel resistant cell lines were maintained in their respective media with addition of 10 nM paclitaxel.

### Example 1

### Solubility of benzimidazoles in DMSO/NMP/TWEEN-80/CrEL

100 mg of albendazole was formulated in either a carrier containing 0.5 mL DMSO, 1.5 mL NMP, 1.0 mL TWEEN®-80, and 1.0 mL CrEL or 4 mL DMSO. 4 samples of each formulation were prepared. The samples with the DMSO/NMP/TWEEN®-80/CrEL solutions were marked tubes 1-4. The samples with only DMSO were marked tubes 5-8. The solutions were diluted with saline one fold (tubes 1 and 5), three fold (tubes 2 and 6), five fold (tubes 3 and 7), and ten fold (tubes 4 and 8). The samples were evaluated visually.

Upon dilution with saline, compound formulated in DMSO alone started to precipitate but remained in suspension. Compound formulated in the DMSO, NMP, and TWEEN® remained as an emulsion.

### Example 2

### Cytotoxicity of benzimidazoles in DMSO/NMP/TWEEN®-80/CrEL

Materials and Methods

Drug Formulation

Carrier for benzimidazole drugs was prepared by mixing DMSO (12.5%), NMP (37.5%), TWEEN®-80 (25%) and Cremophor® EL (25%) in a 50 ml FALCON® tube with frequent vortex. The resulting mixture is stable and can be stored in room temperature. Physically stable refers to a microemulsion that remains in suspension, without precipitation, for one to three days. Drug solution for injection was freshly prepared during each treatment day.

PC3M and PC3MLN4 cells were treated with vehicle, fenbendazole in DMSO, and fenbendazole in DMSO/NMP/TWEEN®-80/CrEL in varying doses for 72 hours. Cell viability assays using Cyquant reagent (Invitrogen, C7026) were performed as described in the literature. 1-4×10⁵ cells were seeded in 96 well plates overnight, before treated with either vehicle or benzimidazole solubilized in DMSO alone or in DMSO (12.5%)/NMP (27.5%)/TWEEN®-80 (25%)/Cr-EL (25%) for 72 hours. Cells were harvested by removing the media, washing once with phosphate buffered saline (PBS) and freezing at −80° C. for overnight. After thawing to room temperature, Cyquant reagent were prepared and added to the wells and the fluorescence intensity were measured with a spectrometer at 485 nm. Percent control was calculated by dividing the reading from drug-treated cells with the reading from vehicle-treated cells.

Results

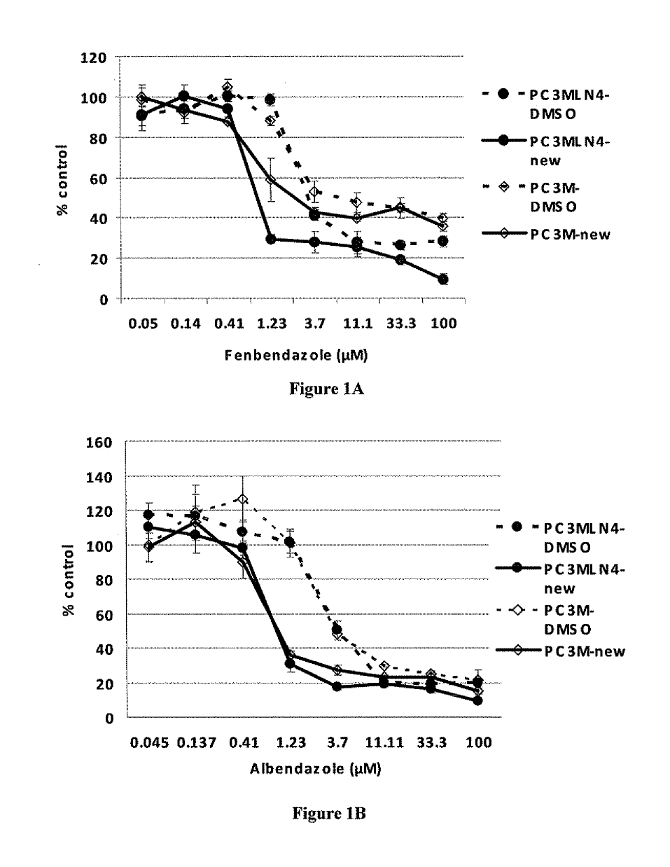

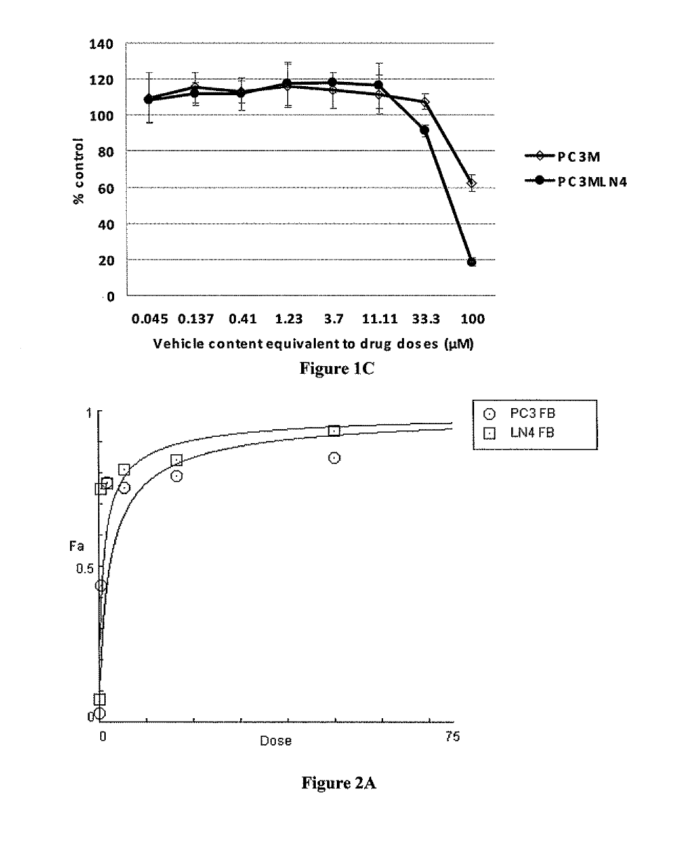

The results are shown in FIGS. 1A and 1B. FIG. 1A compares the cytotoxicity of fenbendazole in DMSO alone or in DMSO/NMP/TWEEN®-80/CrEL against PC3M and PC3MLN4 cells as a function of concentration. Fenbendazole in the combination carrier exhibited greater cytotoxicity against PC3MLN4 cells than in DMSO alone. Further, fenbendazole in the combination carrier exhibited greater toxicity against metastatic PC3MLN4 cells than against PC3M cells.

FIG. 1B compares the cytotoxicity of albendazole in DMSO alone or in DMSO/NMP/TWEEN®-80/CrEL against PC3M and PC3MLN4 cells as a function of concentration. Albendazole in the combination carrier exhibited greater cytotoxicity against PC3MLN4 cells than in DMSO alone. Further, albendazole in the combination carrier exhibited greater cytotoxicity against metastatic PC3MLN4 cells than against PC3M cells.

FIG. 1C shows cell viability as a function of carrier concentration (without drug). FIG. 1C shows that the combination carrier exhibited little or no cytotoxicity at concentrations up to about 35 μM.

Table 1 shows the ED₅₀ for the benzimidazole drugs fenbendazole, albendazole, and oxibendazole, in the combination carrier compared to DMSO alone. ED₅₀ values were calculated from dose-effect analysis performed with Compusyn using data from the Cyquant cell viability assays.

TABLE 1
ED₅₀ values for benzimidazoles in
the combination carrier and DMSO

| ED₅₀ (μM) | |
|---|---|
| PC3M | PC3MLN4 |

| | DMSO | new | DMSO | new |
|---|---|---|---|---|
| Fenbendazole | 5.40 | 2.60 | 1.93 | 0.91 |
| Albendazole | 4.84 | 1.15 | 4.68 | 0.58 |
| Oxibendazole | 8.82 | 1.21 | 4.12 | 2.04 |

As shown in Table 1, benzimidazoles formulated in the combination carrier exhibited greater cytotoxicity against PC3M and PC3MLN4 cells compared to benzimidazoles in DMSO only. Moreover, benzimidazoles formulated in the combination carrier exhibit greater cytotoxicity against cell lines prone to metastases (e.g., PC3MLN4) than cell lines that do not metastasize (e.g., PC3M).

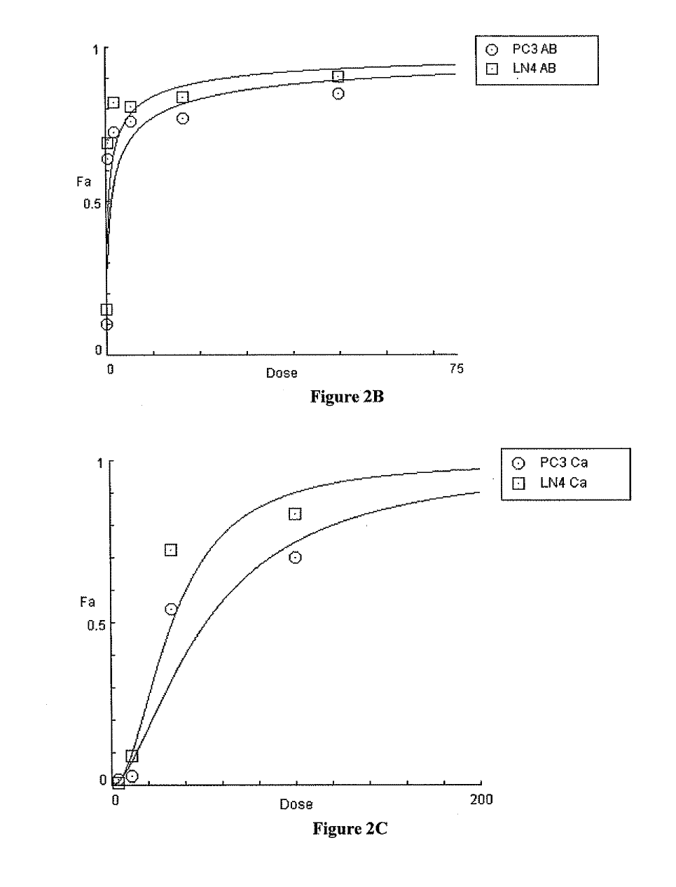

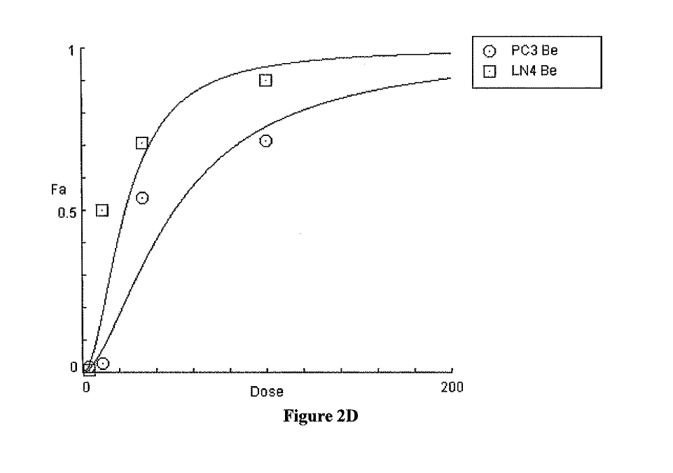

FIGS. 2A-D shows the cytotoxicity of fenbendazole (FIG. 2A), albendazole (FIG. 2B), carbendazim (FIG. 2C), benomyl (FIG. 2D), and the combination carrier against PC3M and PC3MLN4 cells. The active agents were formulated in the combination carrier. The cells were treated for 72 hours. Percent control was calculated by dividing the fluorescent reading (Cyquant assay) from drug-treated cells with those from vehicle-treated cells, indicating percentage of cell survival.

FIGS. 2A-D show that at lower doses (e.g., approximately 1 μM), the benzimidazole exhibits greater cytotoxicity against cell lines prone to metastases than cell lines less prone to metastases. This suggests that metastatic tumors can be treated with lower doses of an antihelmintic benzimidazole than tumors less prone to metastasis. This also means that a higher dosage can be utilized for greater kill of metastatic cells.

Table 2 shows the ED₅₀ values for several benzimidazoles as a function of cancer cell line. ED₅₀ values were calculated from dose-effect analysis performed with Compusyn using data from the Cyquant cell viability assays. All benzimidazoles drugs were solubilized in the combination carrier. NT indicates the compound was not toxic at all doses tested.

TABLE 2
ED₅₀ of various benzimidazoles as a function of cancer cell line.

| | ED₅₀ (μM) | | |
|---|---|---|---|
| Compound | PC3M | PC3MLN4 | AT6.1 |
| Fenbendazole | 1.15 | 0.91 | 0.53 |
| Albendazole | 1.21 | 0.58 | 0.85 |
| Mebendazole | 1.21 | 0.70 | 0.57 |
| Flubendazole | 1.43 | 0.67 | 1.40 |
| Oxibendazole | 4.04 | 2.04 | 1.30 |
| Benomyl | 49.8 | 23.4 | 30.2 |
| Carbendazim | 51.1 | 32.9 | 31.9 |
| Thiophanate methyl | 68.5 | 41.8 | NT |
| Thiabendazole | 127.8 | 60.7 | NT |

As shown in Table 2, the benzimidazoles exhibited greater cytotoxicity against metastatic cells lines (PC3MLN4) than non-metastatic cell lines (PC3M).

These cytotoxic effects are mediated in part by an induction of mitotic arrest, as shown using histone H3 phospho-Serine-28 as a marker of G2/M arrest Table 3 shows the normalized mitotic index of various benzimidazoles at a concentration of 1 μM.

TABLE 3
Normalized mitotic index for various benzimidazoles

| | Normalized mitotic index at 1 μM | | |
|---|---|---|---|
| Compound | PC-3MLN4 | PC-3M | AT6.1 |
| Paclitaxel | 2.34 | 2.43 | 2.10 |
| Albendazole | 1.84 | 1.78 | 1.32 |
| Flubendazole | 2.08 | 1.87 | 1.37 |
| Mebendazole | 1.83 | 1.86 | 1.46 |
| Fenbendazole | 2.27 | 1.86 | 1.54 |
| Oxibendazole | 1.92 | 1.75 | 1.61 |
| Benomyl | 0.99 | 0.91 | 1.08 |
| Carbendazim | 1.00 | 0.91 | 1.08 |
| Thiophanate methyl | 0.95 | 0.95 | 0.96 |
| Thiabendazole | 0.99 | 0.98 | 0.96 |

The mitotic index for each agent was normalized against those obtained from vehicle-treated cells, and compared with the activity of paclitaxel, a known inducer of mitotic arrest. In all three prostate cancer cells tested, fenbendazole, albendazole, mebendazole, flubendazole and oxibendazole showed potent induction of mitotic arrest (value greater than 1.00), with induction in the metastatic PC-3MLN4 cells. Interestingly, the potency of mitotic arrest induced by these compounds does not correlate directly with the degree of cell growth inhibition. For instance, treatment of PC-3MLN4 cells with 1 μM fenbendazole (ED50 0.9 μM) resulted in the highest induction of mitotic arrest (mitotic index 2.27); however, treatment with albendazole which has a lower ED50 (0.58 μM) and oxibendazole which has a higher ED50 (2.04 μM) exhibited mitotic index in the range of 1.8-2.0.

### Example 3

### Benzimidazole Drug-Induced Apoptosis is Higher in PC3MLN4 than in PC3M Cells

Materials and Methods

PC3M cells were treated with (A) combination carrier, (B) 1 μM fenbendazole, (C) 1 μM albendazole or (D) 1 μM mebendazole for 72 hours. All benzimidazole drugs were solubilized in the combination carrier. After 72 hours, the cells were analyzed by annexin V/7-AAD staining.

Annexin V/7-amino-actinomycin D labeling was done according to the manufacturer's instructions (BD Pharmingen, 559763) and samples were analyzed by flow cytometry. Briefly, the cells were treated for 72 hours with either vehicle or 1 μM benzimidazole drugs, solubilized in the combination carrier. Cells were trypsinized and washed with PBS before resuspending in assay binding buffer. Annexin V and 7-amino-actinomycin labeling was performed at room temperature for 15 minutes before analysis by flow cytometry (BD FACScan). Cells positively stained for annexin V in the lower right quadrant and upper right quadrant indicate early and late apoptosis, respectively.

PC3MLN4 cells were treated with (E) vehicle, (F) 1 μM fenbendazole, (G) 1 μM albendazole or (H) 1 μM mebendazole for 72 hours. All benzimidazole drugs were solubilized in the combination carrier. After 72 hours, the cells were analyzed by annexin V/7-AAD staining. The results are shown in FIGS. 3A-3H. Cells positively stained for annexin V in the lower right quadrant and upper right quadrant indicate early and late apoptosis, respectively.

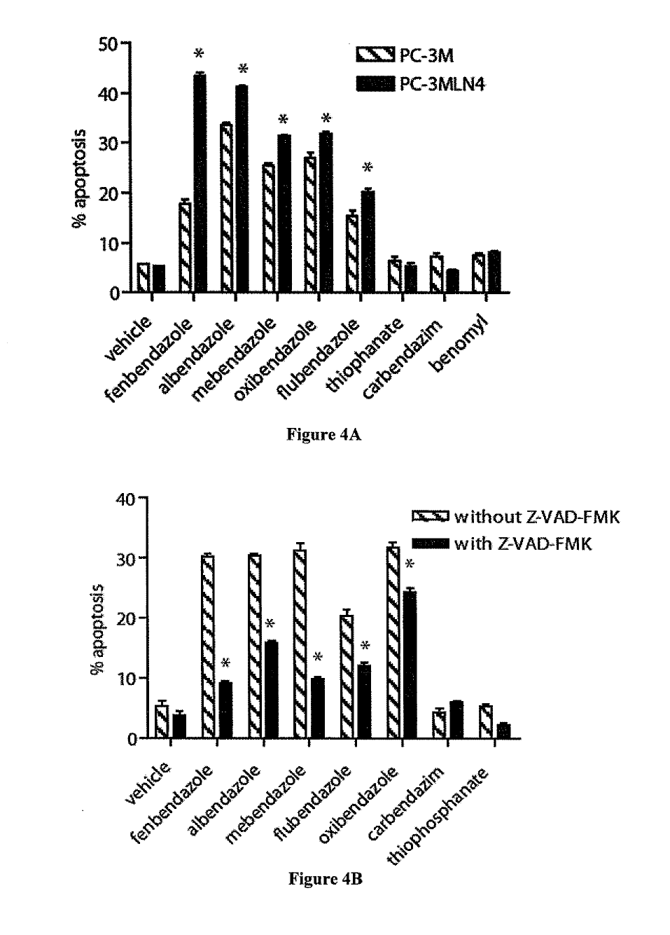

Compared to the vehicle treated cells, benzimidazole treatment results in apoptosis in both PC3M and PC3MLN4 cells, but with a higher degree in PC3MLN4 than in PC3M cells. The results are shown in FIG. 4A. In PC3MLN4 cells, treatment with fenbendazole induced 31.4% apoptotic cells (22.8% in PC3M); albendazole induced 29.7% apoptotic cells (22% in PC3M), and mebendazole induced 17.6% apoptotic cells in PC3MLN4 cells (16.8% in PC3M). These effects were significantly reduced when PC-3MLN4 cells were concurrently treated with a caspase-3 inhibitor (z-VAD-FMK) (FIG. 4B). Taken together, fenbendazole, albendazole, mebendazole, flubendazole and oxibendazole all demonstrate cytotoxic activity against highly metastatic prostate cancer cells in vitro via induction of apoptosis.

### Example 4

### Benzimidazole Drugs Reduce Tumor Burden and Increase Survival of Metastatic Prostate Cancer Cell-Bearing Animals

Materials and Methods

The in vivo efficacy of albendazole (ABZ) (50 and 100 mg/kg), mebendazole (MBZ) (50 and 100 mg/kg) and oxibendazole (OXB) (50 g/kg) were tested in an intravenous AT6.1 Dunning rat prostate carcinoma model.

Inbred male nu/nu mice age 6-8 weeks were obtained from Massachusetts General Hospital (Boston, Mass.). The mice were virus antibody free, age and weight matched for experimental use and fed with a balanced rodent diet ad libitum. AT6.1 Dunning rat prostate cells were maintained as described above. 1×10⁴ log-phase cells were injected via tail vein in 0.1 mL Hanks solution. Vehicle or drug treatment was started on day 5 post inoculation and given three times a week (Monday, Wednesday and Friday) via intraperitoneal injection in 0.5 mL volume. Benzimidazole drugs were prepared in the combination carrier [DMSO (12.5%), NMP (37.5%), TWEEN®-80 (25%) and CrEL (25%)] and diluted in saline. Vehicle control was prepared by diluting the combination carrier with saline.

During the course of treatment, the mice were monitored for toxicity by measuring body weight. Treatment was continued until the animals showed signs of morbidity defined by significant loss of body weight, difficulty in breathing and hunched posture. Animals were euthanized with CO₂ and tissues were collected for further analysis. The animals and experiments used in these studies were approved by the institutional animal care committee according to Children's Hospital Boston ARCH guidelines.

Results

Survival

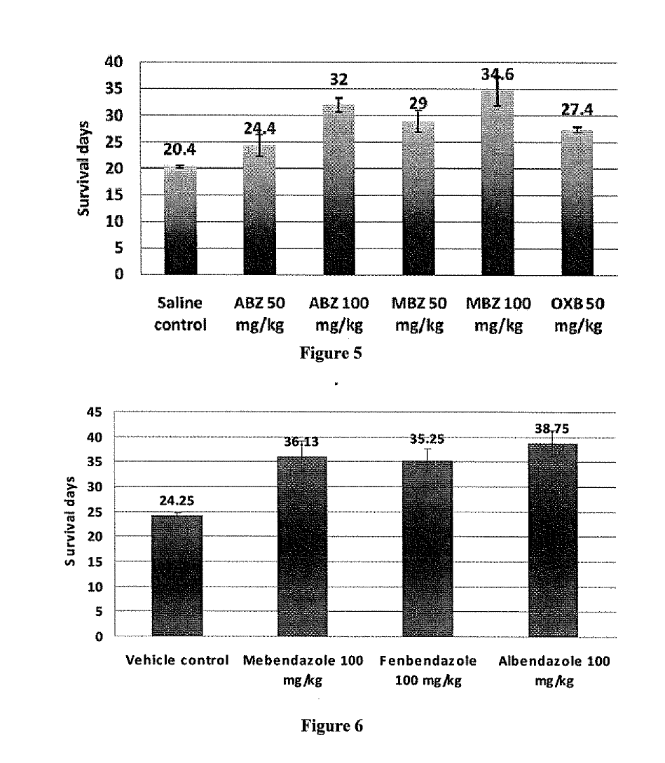

The number of day survival for each treatment was compared to the vehicle control group. The results are shown in FIG. 5. Rats given the combination carrier only had an average survival time of 20.4 days. Rats administered albendazole at a dose of 50 mg/kg and 100 mg/kg had an average survival time of 24.4 and 32 days, respectively. Rats administered mebendazole at a dose of 50 mg/kg and 100 mg/kg has an average survival time of 29 and 34.6 days, respectively. Rats administered oxibendazole at a dose of 50 mg/kg had an average survival time of 27.4 days.

The survival rates of albendazole and mebendazole were compared to the survival rates for fenbendazole. The results are shown in FIG. 6. mebendazole (100 mg/kg), fenbendazole (100 mg/kg), and albendazole (100 mg/kg) exhibited survival rates of 36.13, 35.25, and 38.75 days, respectively, compared to the control (24.25 days).

Compared to the vehicle-treated group, mice receiving fenbendazole, albendazole or mebendazole showed significantly increased survival times. To distinguish differences in survival between these groups, the percent increase in life span (ILS %), defined as the percent increase in the mean time to death of drug-treated animals over the corresponding time to death for vehicle-treated mice, was calculated. The results reveal that the benzimidazoles significantly increase post-metastatic animal viability, with ILS %: fenbendazole (45.3%), albendazole (59.7%) and mebendazole (48.9%). These data demonstrate that multiple members of the benzimidazole family have potent activity in inhibiting the growth of disseminated prostate cancer and in extending the survival of mice bearing these tumors.

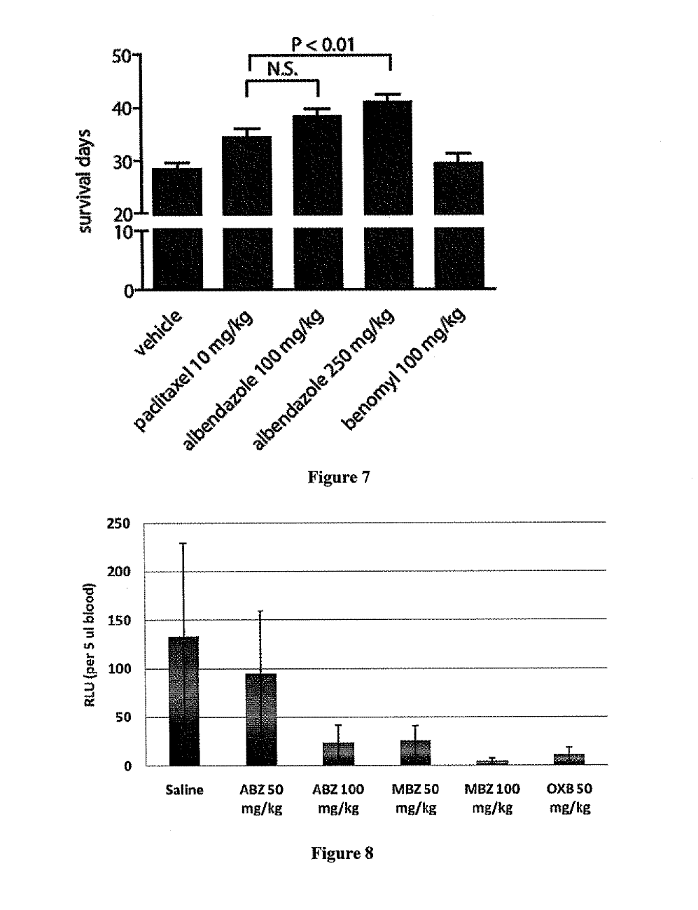

The survival times in mice treated with vehicle, paclitaxel, albendazole (100 mg/kg and 250 mg/kg), and benomyl (100 mg/kg) were also compared. The results are shown in FIG. 7. Paclitaxel and albendazole-treated mice showed increased survival relative to the vehicle-treated group. The increase provided by treatment with albendazole at 250 mg/kg (ILS % 64) was significantly greater than that seen in animals treated with paclitaxel at 10 mg/kg (ILS % 30.2) (P<0.01). Confirming the in vitro data, benomyl showed little or no extension of lifespan of tumor-bearing animals in vivo (ILS % 7.5).

Tumor Burden

Blood Gaussia luciferase assay was performed as described in the literature. Briefly, 10-20 μL peripheral blood was collected from the animals once a week via the retro-orbital vein. Secreted GAR luciferase activity was measured using a luminometer by adding 100 μL of 100 μM coelentrazine (Prolume, Nanolight, #303) to 5 μL blood and 2 μL of 20 mM EDTA. Relative luciferase reading from the blood collected represents the tumor burden in each animal. The reading was performed with blood collected at day 19 post inoculation when all mice were still alive. The results are shown in FIG. 8.

Benzimidazole drugs reduced tumor burden compared to the combination carrier in saline (control) (see FIG. 8). The reduction in tumor burden was most pronounced for the formulation containing 100 mg/kg mebendazole.

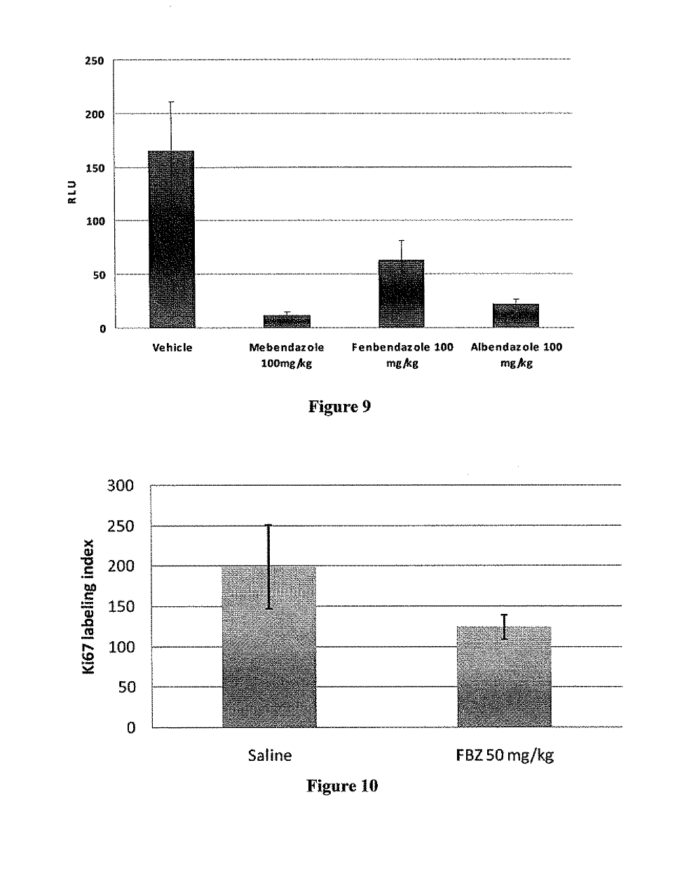

Tumor burden reduction of mebendazole (100 mg/kg), fenbendazole (100 mg/kg), and albendazole (100 mg/kg), administered 3 times a day until day 22, in blood collected at day 22 post inoculation, compared to the control is shown in FIG. 9. Mebendazole, febendazole, and albendazole showed a significant reduction in tumor burden compared to the control, with mebendazole and albendazole showing more reduction than fenbendazole.

### Example 5

### In Vivo Fenbendazole Treatment Decreases Tumor Cell Proliferation and Increases Tumor Cell Death

### Materials and Methods

Tumor cells in the lungs of rats treated with saline and 50 mg/kg fenbendazole (formulation in the combination carrier) were analyzed by Ki-67 and caspase-3 antibody staining. Tissues were collected and fixed in formalin before processed for paraffin embedding. Tissue sections were then processed for Ki-67 and caspase-3 antibody staining, using the protocol established at Dana Farber Cancer Institute/Harvard Cancer Center Research Pathology Core. At least five random 200× magnification images were photographed from each animal. Distinct and strong nuclear Ki67 staining of tumor cells were counted using Image J software (NIH, Bethesda, D.C.).

Results

Tumor sections from vehicle-treated animals showed significant more distinct, strong nuclear staining of KI67, a marker of proliferation, when compared to the tumor sections from fenbendazole-treated animals, indicating that drug treatment resulted in less proliferation of tumor cells.

The effect of fenbendazole (50 mg/kg) on tumor cell proliferation and cell death, compared to a saline control, is shown in FIG. 10. Compared to the control, administration of fenbendazole formulated in the combination carrier showed a marked reduction in tumor cell proliferation compared to the control.

In the AT6.1 model, there was a marked reduction of luciferase activity in compound-treated groups when compared to the control group. In addition, Ki-67 and caspase-3 staining showed that anti-parasitic compounds preferentially target tumor cells by inducing apoptosis. These results were confirmed in vitro using propidium iodide and annexin V staining to analyze DNA distribution and apoptosis, respectively. These anti-parasitic compounds induced G₂-M cell cycle arrest at 6 hours post treatment, which led to a significant increase of apoptotic cell population after 72 hours post treatment.

### Example 6

### Combination Treatment of Benzimidazole Drugs with Paclitaxel Results in Enhanced Anti-Tumor Effects In Vitro

Materials and Methods

Cell growth inhibition assays were performed using Cyquant reagent as described above. Drug interactions were quantitated by median-dose effect analysis and combination index values as well as dose reduction index were derived using CompuSyn software (ComboSyn, Inc.), as described previously in the literature. CI values of <1, =1, and >1 indicate synergism, additivity and antagonism between the drugs, respectively.

Combination index plots for combination treatment of (A) fenbendazole, (B) albendazole and (C) mebendazole with paclitaxel were generated using CompuSyn software. All benzimidazole drugs were prepared using the combination carrier and diluted with culture medium prior to treatment.

Results

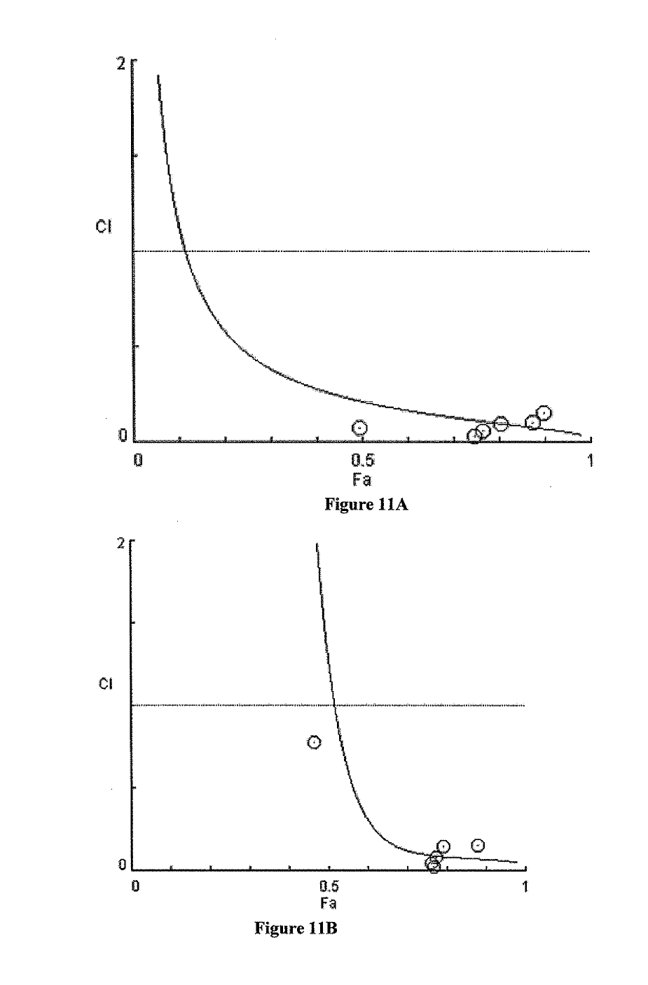

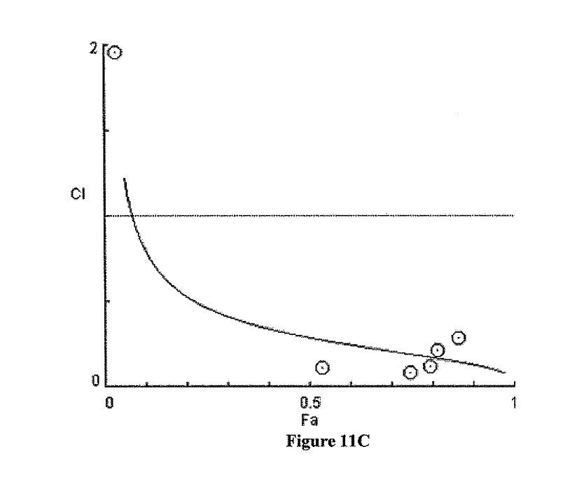

The results are shown in FIGS. 11A, 11B and 11C. FIG. 11A shows combination index plot with fraction affected (Fa) on the x-axis and the combination index (CI) on the Y-axis. The graph line indicates the combination index value at each point of fraction affected. At a given desired fraction affected, the combination treatment suggests synergism when CI value is higher than one. In FIG. 11A, the combination of fenbendazole with paclitaxel results in synergistic effects in fraction affected above 15%. FIG. 11B shows that combination index of albendazole with paclitaxel only results in synergisms when fraction affected is above 50%. FIG. 11C indicates that combination treatment of mebendazole with paclitaxel is synergistic in most percent fraction affected.

Table 4 shows the dose reduction of the benzimidazole and paclitaxel when the drugs are administered together.

TABLE 4
Dose reduction of paclitaxel (Tax) and benzimidazoles
(fenbendazole (FBZ) and albendazole (ABZ))

| | | | Dose Tax | Dose FBZ | DRI | DRI |
|---|---|---|---|---|---|---|
| Fa | Drug/Combo | CI value | (nM) | (nM) | Tax | FBZ |
| 0.50 | Tax | | 937 | | | |
| | FBZ | | | 6206 | | |
| | Tax-FBZ | 0.21 | 90.8 | 726.7 | 10.3 | 8.5 |
| | | CI | Dose Tax | Dose ABZ | DRI | DRI |
| Fa | Drug/Combo | value | (nM) | (nM) | Tax | ABZ |
| 0.60 | Tax | | 1392 | | | |
| | ABZ | | | 5249 | | |
| | Tax-ABZ | 0.30 | 137.8 | 1117 | 10.1 | 4.7 |
| | | CI | Dose Tax | Dose MBZ | DRI | DRI |
| Fa | Drug/Combo | value | (nM) | (nM) | Tax | MBZ |
| 0.50 | Tax | | 936 | | | |
| | MBZ | | | 17456 | | |
| | Tax-MBZ | 0.287 | 237 | 1899 | 4.4 | 16.4 |

### Example 7

### Benzimidazole Drugs are Effective in Both Paclitaxel-Sensitive and Paclitaxel-Resistant Prostate Cancer Cell Lines

Materials and Methods

The cytotoxic effects of paclitaxel, fenbendazole, and albendazole were compared in paclitaxel-resistant PC3-TR and DU145-TR prostate cancer cells with their paclitaxel-sensitive counterparts, PC3 and DU145 cells. Cells were treated for 72 hours with vehicle, paclitaxel, fenbendazole or albendazole in various doses. Cell viability was measured using Cyquant assay, and percent control was calculated by dividing the fluorescent reading from drug-treated cells with those from vehicle-treated cells, indicating percentage of cell survival.

Results

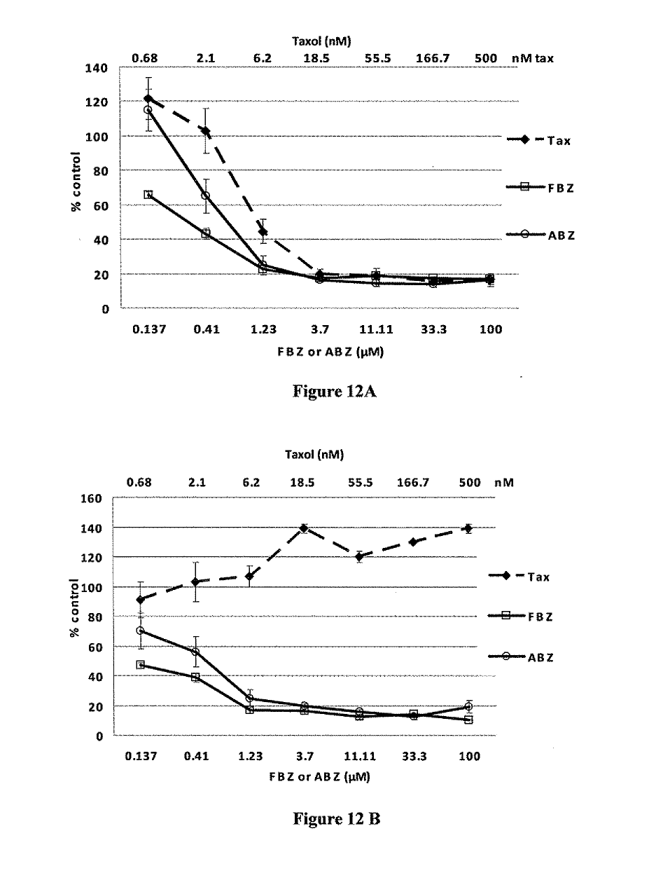

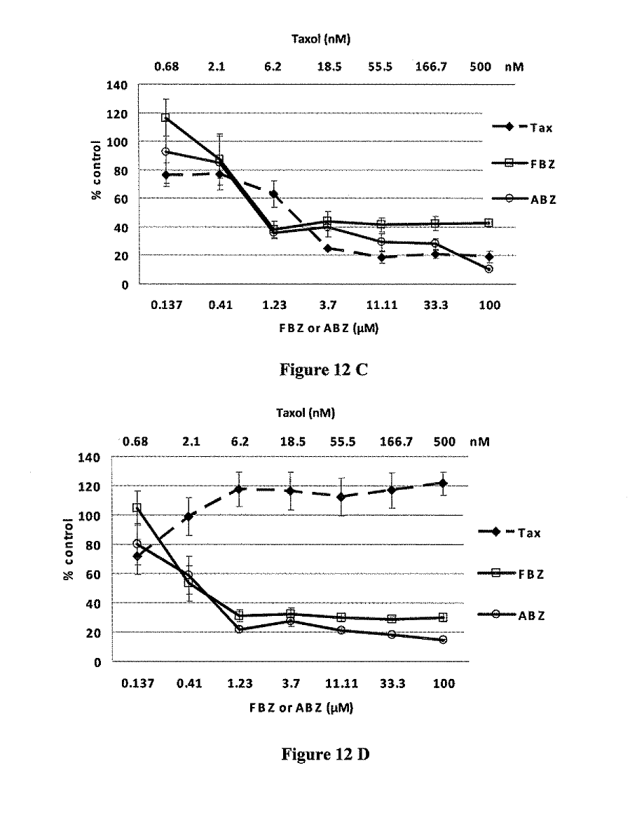

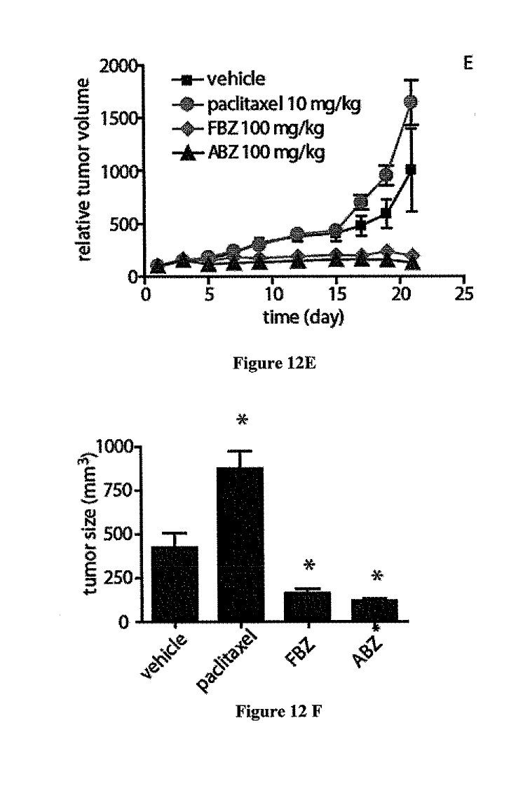

The results are shown in FIG. 12. FIGS. 12A-12D are graphs comparing the cytotoxic effect (percent control) of paclitaxel (♦), fenbendazole (□), and albendazole (∘) against paclitaxel resistant PC3-TR (FIG. 12B) and DU145-TR (FIG. 12D) cell lines and their paclitaxel-sensitive counterparts PC3 (FIG. 12A) and DU 145 (FIG. 12C) cell lines. As expected, the taxol alone was less effective against the PC3-TR and DU145-TR cells lines, but the benzimidazoles were highly effective.

Benzimidazoles retain their anti-tumor activity in these paclitaxel-resistant cells, with higher potency when compared to the parental cells (see Table 4). Cells resistant to microtubule-stabilizing drugs such as the taxanes are more susceptible to microtubule-disrupting agents. To confirm this, human uterine sarcoma MES-SA/Dx5 cells, which are cross-resistant to various chemotherapeutic agents, including the microtubule-disrupting agents vincristine and colchicine, were used. Treatment with benzimidazoles resulted in a significant cytotoxic effect in the multidrug resistant MES-SA/Dx5 cells relative to the parental MES-SA cells (Table 5). This suggests that benzimidazoles may able to overcome single agent or multidrug-resistant hormone refractory prostate cancer cells regardless of the type of microtubule-targeting agents used in the initial round of chemotherapy.

TABLE 5
Comparison of Benzimidazole Treatment in Paclitaxel-Sensitive
and -Resistant Cancer Cell Lines

| | ED₅₀ (μM) | | | | | |
|---|---|---|---|---|---|---|
| | | PC3- | | DU145- | MES- | |
| Compound | PC3 | TR | DU145 | TR | SA | MES-SA/Dx5 |
| Febendazole | 1.57 | 0.32 | 10.7 | 4.21 | 3.04 | 1.13 |
| Albendazole | 4.86 | 0.80 | 2.41 | 1.08 | 1.48 | 0.84 |
| Mebendazole | 2.85 | 0.75 | 5.90 | 2.97 | 1.27 | 0.79 |

Further analysis with PC-3TR and DU145-TR cells reveals that both cells demonstrate different mechanisms of resistance to paclitaxel-mediated cytotoxicity (see Table 6). Treatment with paclitaxel induced mitotic arrest in PC-3TR but not in DU-145TR, suggesting that there could be defects in apoptotic machinery in PC-3TR and in cell cycle arrest machinery in DU145-TR. Nevertheless, induction of apoptosis by benzimidazoles in both PC-3TR and DU145-TR cells indicate that benzimidazole-mediated apoptosis may use different pathways from those utilized by paclitaxel in PC-3TR cells and may be independent of mitotic arrest in DU145-TR.

TABLE 6
Comparison of Benzimidazole Treatment in Paclitaxel-Sensitive
and -Resistant Cancer Cell Lines

| | Normalized mitotic index | | | | | | | |
|---|---|---|---|---|---|---|---|---|
| | (6 hours) | | | | % Apoptotic cells (72 hours) | | | |
| | | | | DU145- | | | | DU145- |
| Compound | PC3 | PC3-TR | DU145 | TR | PC3 | PC3-TR | DU145 | TR |
| Vehicle | 1.00 | 1.00 | 1.00 | 1.00 | 8.1 | 10.1 | 9.6 | 9.0 |
| Paclitaxel | 1.32 | 1.56 | 3.52 | 0.86 | 16.4 | 8.9 | 37.3 | 11.5 |
| (100 nM) | | | | | | | | |
| Fenbendazole | 1.26 | 1.48 | 1.47 | 1.18 | 16.1 | 34.4 | 26 | 43.3 |
| (1 μM) | | | | | | | | |
| Albendazole | 1.27 | 1.56 | 1.93 | 1.13 | 13.2 | 25.8 | 27 | 55.8 |
| (1 μM) | | | | | | | | |
| Mebendazole | 1.27 | 1.35 | 1.96 | 1.12 | 11.9 | 21.5 | 16.4 | 36.7 |
| (1 μM) | | | | | | | | |

In order to dissect the mechanisms underlying the anti-tumor effects of benzimidazoles in these cells, gene expression profiling was done comparing albendazole treated DU-145 and DU-145TR cells. With a 2-fold cut off, several metastases, cell cycle and apoptosis-related genes were found to be differentially regulated in the DU-145TR cells. Genes implicated in endoplasmic reticulum (ER) stress signaling, including activating transcription factor (ATF)-2 and -3, HERPUD1, CHOP (DDIT3), ANKRD1 (CARP) and GADD45alpha were differentially expressed in DU-145TR cells upon treatment with albendazole, suggesting that albendazole-induced apoptosis may involve an ER stress response.

The anti-tumor activity of the benzimidazoles on paclitaxel-resistant cells was confirmed in vivo. PC-3TxR cells were inoculated subcutaneously in the flanks of the nude mice. When the tumors reached approximately 100 mm3 in size, the mice were treated with vehicle, paclitaxel (10 mg/kg), fenbendazole (100 mg/kg) or albendazole (100 mg/kg) three times a week for 3 weeks. These benzimidazoles were prepared in DNTC and diluted with saline prior to injection. Compared to the vehicle treated group, the growth of PC-3TxR tumors in the paclitaxel group was significantly greater, indicating these cells require paclitaxel for optimal growth (FIGS. 12E and F). In contrast, tumors treated with either fenbendazole or albendazole were growth inhibited and remained at approximately 100-150 mm³ in size throughout the study. These data strongly suggest benzimidazoles are effective against paclitaxel-resistant prostate cancer cells in vitro and in vivo.

### Example 8

### Treatment of Metastatic Lung and Bone Cancer

In the lung metastasis model, treatment was initiated 10 days after the animals were inoculated with tumor cells. At this time, the lungs were riddled with metastases. Formulations containing a benzimidazole were injected into the peritoneum three times per week. Half of the control mice died by day 27, only 17 days after the animals started receiving treatments. In contrast, half of the mice treated with fenbendazole were still alive on day 37 and one-third were still alive at day 45. Efficacy studies showed that fenbendazole is equivalent to taxol in prolonging the survival times of the mice.

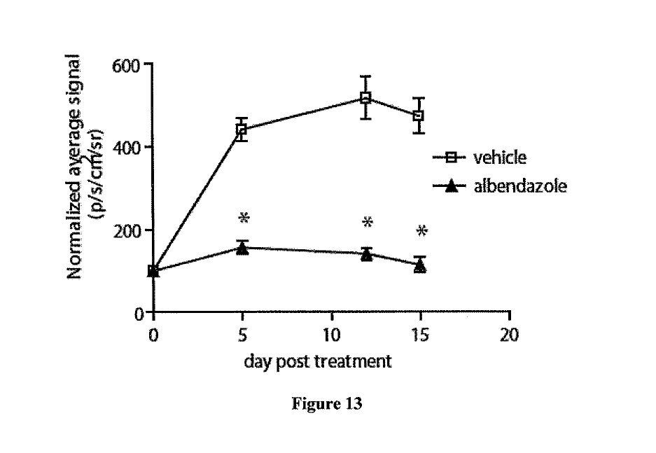

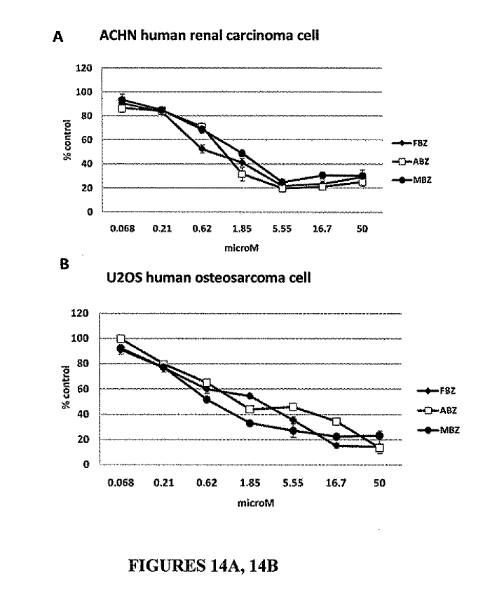

Using the metastatic prostate cancer PC-3MLN4 cells that expressed luciferase, the anti-tumor effects of benzimidazoles in a model of experimental bone metastasis were investigated. Cells were inoculated directly into a single tibia of immunocompromised mice via intraosseous injection. When bone lesions were confirmed to be growing via bioluminescent imaging, mice were randomized and treated with vehicle or with 100 mg/kg albendazole for two weeks. Mice treated with albendazole showed reduced tumor growth compared to those treated with vehicle. There was a significant difference in average luciferase signal between the two groups over the course of treatment (P<0.05) (see FIG. 13).

At the end of study, the anatomical and radiographical characteristics of the bone lesions were evaluated with X-ray and micro-computerized tomography (CT) imaging. Mice treated with vehicle showed extensive osteolysis due to the osteoclastic bone resorption activity of PC-3MLN4 cells. In contrast, the bone integrity was maintained in albendazole-treated mice. Immunohistochemical analysis of these bone lesions further showed a reduction of Ki-67 labeling index (P<0.005) and an increase of apoptotic index, based on activated caspase-3 staining (P<0.005) in the albendazole-treated group. These results demonstrated that the anti-tumor effects of benzimidazoles extend to tumors growing in intraosseuous spaces.

### Example 9

Treatment of Renal Cell Carcinoma Cells, Osteosarcoma Cells, Pancreatic Adenocarcinoma Cells, Fibrosarcoma Cells, Uterine Sarcoma, Breast Cancer Cells, Adenocarcinoma Cells and Non-Small Cell Lung Cancer Cells

Materials and Methods

Cell Lines.

ACHN, U2OS, AsPC-1, BxPC-3, Capan-2, HT1080, MESSA, MCF-7, A549 and 11460 cells were obtained from ATCC. ACHN, U2OS and MCF-7 cells were maintained in DMEM media supplemented with 10% fetal bovine serum and 1% penicillin/streptomycin. AsPC-1 and BxPC-3 cells were maintained in RPMI 1640 supplemented with 10% fetal bovine serum and 1% penicillin/streptomycin. Capan-2 cells were maintained in McCoy's modified media supplemented with 10% fetal bovine serum and 1% penicillin/streptomycin. HT1080 and 11460 cells were maintained in MEM media supplemented with 10% fetal bovine serum and 1% penicillin/streptomycin, while A549 and MESSA cell lines were maintained in F12K media supplemented with 10% fetal bovine serum and 1% penicillin/streptomycin.

Treatment of Cells

The active agents were formulated in the combination carrier. The cells were treated for 72 hours.

Cyquant Cell Viability Assay.

Cell viability was assayed using Cyquant reagent (Invitrogen, C7026). Briefly, 1-4×105 cells were seeded in 96 well plate overnight, before treatment with either vehicle or benzimidazole drugs solubilized in DMSO Alone or

in DMSO (12.5%)/NMP (27.5%)/TWEEN®-80 (25%)/Cr-EL (25%) for 72 hours. Cells were harvested by removing the media, washing once with PBS and freezing in −80° C. for overnight. After thawing to room temperature, Cyquant reagent was prepared and added to the wells and the fluorescence intensity were read with a spectrometer at 485 nm. Percent control was calculated by dividing the fluorescent reading (Cyquant assay) from drug-treated cells with those from vehicle-treated cells, indicating percentage of cell survival.

Results

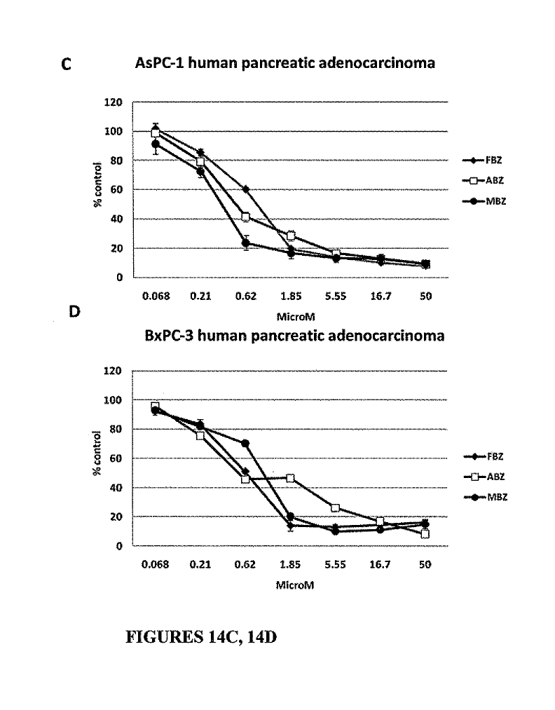

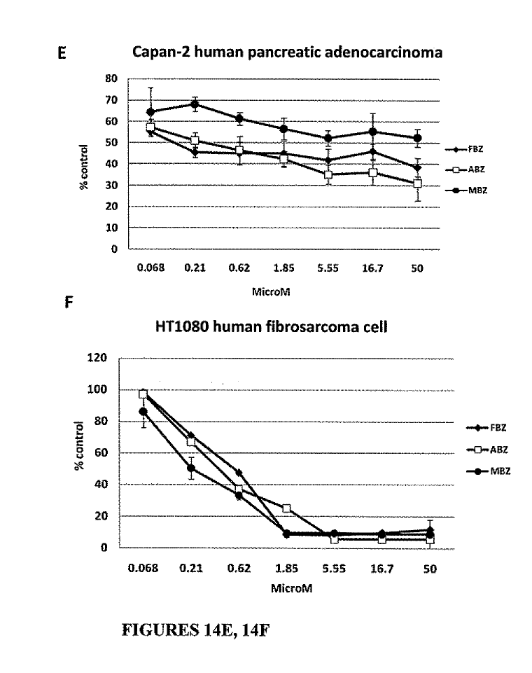

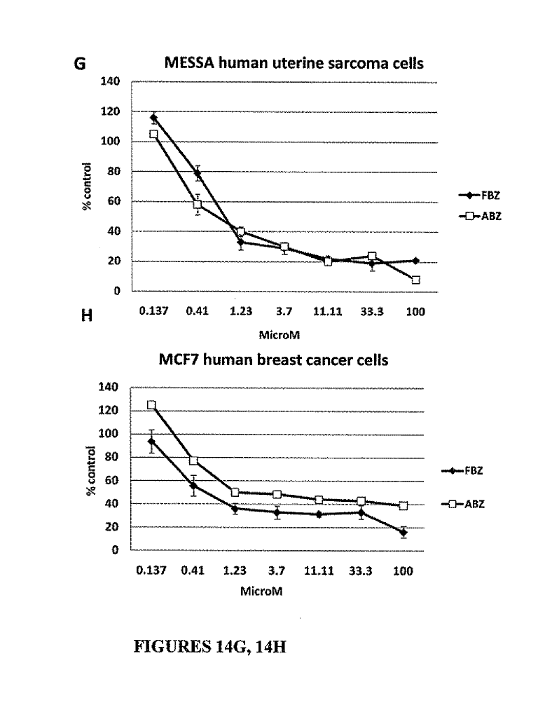

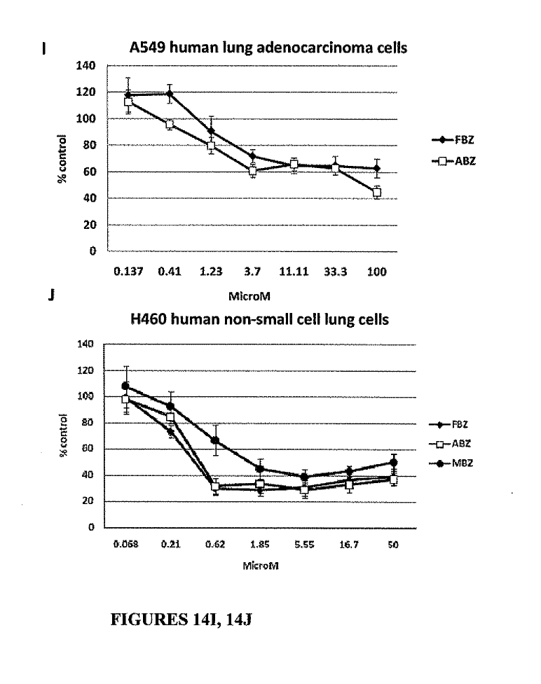

FIGS. 14A-J show the cytotoxicity of fenbendazole, albendazole or mebendazole against various human cancer types including ACHN human renal cell carcinoma cells (FIG. 14A), U2OS human osteosarcoma cells (FIG. 14B), AsPC-1, BxPC-3 and Capan-2 human pancreatic adenocarcinoma cells (FIG. 14C-E), HT1080 human fibrosarcoma cells (FIG. 14F), MESSA human uterine sarcoma (FIG. 14G), MCF-7 human breast cancer cells (FIG. 14H), A549 human lung adenocarcinoma cells (FIG. 14I) and H460 human non-small cell lung cancer cells (FIG. 143). Benzimidazoles are highly effective in treating all cell lines, although to a lesser extent with the pancreatic adenocarcinoma line.

Unless defined otherwise, all technical and scientific terms used herein have the same meanings as commonly understood by one of skill in the art to which the disclosed invention belongs.

Those skilled in the art will recognize, or be able to ascertain using no more than routine experimentation, many equivalents to the specific embodiments of the invention described herein. Such equivalents are intended to be encompassed by the following claims.

1. A pharmaceutical composition for the systemic treatment of metastatic cancer in a patient, the composition comprising one or more benzimidazoles and a pharmaceutically acceptable carrier, wherein the one or more benzimidazoles are present in an effective amount to reduce or prevent the proliferation of the metastatic cancers throughout the patient.

2. The composition of claim 1, wherein the one or more benzimidazoles are selected from the group consisting of fenbendazole, albendazole, flubendazole, oxbendazole, mebendazole, enbendazole, albendazole sulfone, rycobendazole, thiabendazole, oxfendazole, flubendazole carbendazim, benomyl, thiabendazole, derivatives and analogs thereof, prodrugs thereof, and combinations thereof.

3. The composition of claim 2, wherein the benzimidazole is albendazole or a derivative or analog thereof.

4. The composition of claim 2, wherein the benzimidazole is fenbendazole or a derivative or analog thereof.

5. The composition of claim 2, wherein the benzimidazole is mebendazole or a derivative or analog thereof.

6. The composition of claim 1 wherein the dosage is greater than 5 mg/kg, in a dosage unit for treatment of a 70 kg person.

7. The composition of claim 1, wherein the composition further comprises one or more additional active agents.

8. The composition of claim 7, wherein the one or more additional active agents is paclitaxel.

9. The composition of claim 1, wherein the metastatic cancer is metastatic prostate cancer.

10. The composition of claim 9, wherein the prostate cancer has metastasized to the lungs, bone, liver, brain, or combinations thereof.

11. The composition of claim 10, wherein the prostate cancer has metastasized to the bone.

12. The composition of claim 10, wherein the prostate cancer has metastasized to the lung.

13. A formulation for the systemic treatment of metastatic cancer in a patient, the formulation comprising the composition of claim 1, wherein the pharmaceutically acceptable carrier comprises an organic solvent and a surfactant and is in the form of an emulsion, dry particle, or suspension.

14. The formulation of claim 13, wherein the carrier comprises 12.5% DMSO by weight of the carrier, 37.5% NMP by weight of the carrier, 25% TWEEN®-80 by weight of the carrier, and 25% Cremophor EL by weight of the carrier.

15. The formulation of claim 13, wherein the benzimidazole is a single stereoisomer or a mixture of stereoisomers.

16. A method for treating metastatic cancer in a patient, the method comprising administering an effective amount of the composition of claim 1 or the formulation of claim 13 to inhibit proliferation or induce apoptosis of metastatic cancer cells.

17. The method of claim 16 wherein the cancer is selected from the group consisting of prostate cancer cells, renal cell carcinoma cells, osteosarcoma cells, pancreatic adenocarcinoma cells, fibrosarcoma cells, uterine sarcoma, breast cancer cells, adenocarcinoma cells and non-small cell lung cancer cell.

18. The method of claim 16, wherein the metastatic cancer is metastatic prostate cancer.

19. The method of claim 16, wherein the metastatic cancer is in the lungs, bone, liver, brain, or combinations thereof.

20. The method of claim 19, wherein the metastatic cancer is in the bone.

21. The method of claim 19, wherein the metastatic cancer is in the lung.

22. The method of claim 16 wherein the cancer cells are taxane-resistant.

23. A method for screening for compounds which preferentially inhibit proliferation or viability of metastatic cancer cells comprising administering the compound to an animal having been injected intravenously with tumor cells and allowed to grow prior to the compound to be screened being administered.

24. An animal model to screen for compounds preferentially inhibiting proliferation or viability of metastatic cancer cells comprising an animal having been injected intravenously with metastatic tumor cells and allowed to grow tumors prior to the compound to be screened being administered.

25. A pharmaceutical composition for the systemic treatment of metastatic cancer in a patient, the composition comprising one or more compounds identified by the screen of claim 23 and a pharmaceutically acceptable carrier, wherein the one or more compounds are present in an effective amount to reduce or prevent the proliferation of the metastatic cancers throughout the patient.
